SEQUENCE LISTING (114 sequences)

SEQ ID NO: 1 - protein, 4 aa, Artificial sequence
  antibody binding epitop of TNF-related apoptosis-inducing ligand (TRAIL)
STVQ

SEQ ID NO: 2 - protein, 7 aa, Artificial sequence
  antibody binding epitop of TNF-related apoptosis-inducing ligand (TRAIL)
ISPLVRE

SEQ ID NO: 3 - protein, 6 aa, Artificial sequence
  antibody binding epitop of TNF-related apoptosis-inducing ligand (TRAIL)
ERGPQR

SEQ ID NO: 4 - protein, 6 aa, Artificial sequence
  antibody binding epitop of TNF-related apoptosis-inducing ligand (TRAIL)
GRSNTL

SEQ ID NO: 5 - protein, 7 aa, Artificial sequence
  antibody binding epitop of TNF-related apoptosis-inducing ligand (TRAIL)
LSSPNSK

SEQ ID NO: 6 - protein, 8 aa, Artificial sequence
  antibody binding epitop of TNF-related apoptosis-inducing ligand (TRAIL)
KALGRKIN

SEQ ID NO: 7 - protein, 6 aa, Artificial sequence
  antibody binding epitop of TNF-related apoptosis-inducing ligand (TRAIL)
FRFQEE

SEQ ID NO: 8 - protein, 6 aa, Artificial sequence
  antibody binding epitop of TNF-related apoptosis-inducing ligand (TRAIL)
IKENTK

SEQ ID NO: 9 - protein, 6 aa, Artificial sequence
  antibody binding epitop of TNF-related apoptosis-inducing ligand (TRAIL)
VTNEHL

SEQ ID NO: 10 - protein, 4 aa, Artificial sequence
  antibody binding epitop of TNF-related apoptosis-inducing ligand (TRAIL)
RGRS

SEQ ID NO: 11 - protein, 4 aa, Artificial sequence
  antibody binding epitop of TNF-related apoptosis-inducing ligand (TRAIL)
GRSN

SEQ ID NO: 12 - protein, 4 aa, Artificial sequence
  antibody binding epitop of TNF-related apoptosis-inducing ligand (TRAIL)
RSNT

SEQ ID NO: 13 - protein, 4 aa, Artificial sequence
  antibody binding epitop of TNF-related apoptosis-inducing ligand (TRAIL)
SNTL

SEQ ID NO: 14 - protein, 4 aa, Artificial sequence
  antibody binding epitop of TNF-related apoptosis-inducing ligand (TRAIL)
NTLS

SEQ ID NO: 15 - protein, 14 aa, Artificial sequence
  antibody binding epitop of TNF-related apoptosis-inducing ligand (TRAIL)
FRFQEEIKENTKND

SEQ ID NO: 16 - protein, 4 aa, Artificial sequence
  antibody binding epitop of TNF-related apoptosis-inducing ligand (TRAIL)
TLSS

SEQ ID NO: 17 - protein, 4 aa, Artificial sequence
  antibody binding epitop of TNF-related apoptosis-inducing ligand (TRAIL)
LSSP

SEQ ID NO: 18 - protein, 4 aa, Artificial sequence
  antibody binding epitop of TNF-related apoptosis-inducing ligand (TRAIL)
SSPN

SEQ ID NO: 19 - protein, 4 aa, Artificial sequence
  antibody binding epitop of TNF-related apoptosis-inducing ligand (TRAIL)
SPNS

SEQ ID NO: 20 - protein, 4 aa, Artificial sequence
  antibody binding epitop of TNF-related apoptosis-inducing ligand (TRAIL)
PNSK

SEQ ID NO: 21 - protein, 4 aa, Artificial sequence
  antibody binding epitop of TNF-related apoptosis-inducing ligand (TRAIL)
NSKN

SEQ ID NO: 22 - protein, 4 aa, Artificial sequence
  antibody binding epitop of TNF-related apoptosis-inducing ligand (TRAIL)
SKNE

SEQ ID NO: 23 - protein, 4 aa, Artificial sequence
  antibody binding epitop of TNF-related apoptosis-inducing ligand (TRAIL)
KNEK

SEQ ID NO: 24 - protein, 4 aa, Artificial sequence
  antibody binding epitop of TNF-related apoptosis-inducing ligand (TRAIL)
NEKA

SEQ ID NO: 25 - protein, 4 aa, Artificial sequence
  antibody binding epitop of TNF-related apoptosis-inducing ligand (TRAIL)
EKAL

SEQ ID NO: 26 - protein, 4 aa, Artificial sequence
  antibody binding epitop of TNF-related apoptosis-inducing ligand (TRAIL)
KALG

SEQ ID NO: 27 - protein, 4 aa, Artificial sequence
  antibody binding epitop of TNF-related apoptosis-inducing ligand (TRAIL)
ALGR

SEQ ID NO: 28 - protein, 4 aa, Artificial sequence
  antibody binding epitop of TNF-related apoptosis-inducing ligand (TRAIL)
LGRK

SEQ ID NO: 29 - protein, 4 aa, Artificial sequence
  antibody binding epitop of TNF-related apoptosis-inducing ligand (TRAIL)
GRKI

SEQ ID NO: 30 - protein, 4 aa, Artificial sequence
  antibody binding epitop of TNF-related apoptosis-inducing ligand (TRAIL)
RKIN

SEQ ID NO: 31 - protein, 4 aa, Artificial sequence
  antibody binding epitop of TNF-related apoptosis-inducing ligand (TRAIL)
FRFQ

SEQ ID NO: 32 - protein, 5 aa, Artificial sequence
  antibody binding epitop of TNF-related apoptosis-inducing ligand (TRAIL)
RGRSN

SEQ ID NO: 33 - protein, 5 aa, Artificial sequence
  antibody binding epitop of TNF-related apoptosis-inducing ligand (TRAIL)
GRSNT

SEQ ID NO: 34 - protein, 5 aa, Artificial sequence
  antibody binding epitop of TNF-related apoptosis-inducing ligand (TRAIL)
RSNTL

SEQ ID NO: 35 - protein, 5 aa, Artificial sequence
  antibody binding epitop of TNF-related apoptosis-inducing ligand (TRAIL)
SNTLS

SEQ ID NO: 36 - protein, 5 aa, Artificial sequence
  antibody binding epitop of TNF-related apoptosis-inducing ligand (TRAIL)
NTLSS

SEQ ID NO: 37 - protein, 5 aa, Artificial sequence
  antibody binding epitop of TNF-related apoptosis-inducing ligand (TRAIL)
TLSSP

SEQ ID NO: 38 - protein, 5 aa, Artificial sequence
  antibody binding epitop of TNF-related apoptosis-inducing ligand (TRAIL)
LSSPN

SEQ ID NO: 39 - protein, 5 aa, Artificial sequence
  antibody binding epitop of TNF-related apoptosis-inducing ligand (TRAIL)
SSPNS

SEQ ID NO: 40 - protein, 5 aa, Artificial sequence
  antibody binding epitop of TNF-related apoptosis-inducing ligand (TRAIL)
SPNSK

SEQ ID NO: 41 - protein, 5 aa, Artificial sequence
  antibody binding epitop of TNF-related apoptosis-inducing ligand (TRAIL)
PNSKN

SEQ ID NO: 42 - protein, 5 aa, Artificial sequence
  antibody binding epitop of TNF-related apoptosis-inducing ligand (TRAIL)
NSKNE

SEQ ID NO: 43 - protein, 5 aa, Artificial sequence
  antibody binding epitop of TNF-related apoptosis-inducing ligand (TRAIL)
SKNEK

SEQ ID NO: 44 - protein, 5 aa, Artificial sequence
  antibody binding epitop of TNF-related apoptosis-inducing ligand (TRAIL)
KNEKA

SEQ ID NO: 45 - protein, 5 aa, Artificial sequence
  antibody binding epitop of TNF-related apoptosis-inducing ligand (TRAIL)
NEKAL

SEQ ID NO: 46 - protein, 5 aa, Artificial sequence
  antibody binding epitop of TNF-related apoptosis-inducing ligand (TRAIL)
EKALG

SEQ ID NO: 47 - protein, 5 aa, Artificial sequence
  antibody binding epitop of TNF-related apoptosis-inducing ligand (TRAIL)
KALGR

SEQ ID NO: 48 - protein, 5 aa, Artificial sequence
  antibody binding epitop of TNF-related apoptosis-inducing ligand (TRAIL)
ALGRK

SEQ ID NO: 49 - protein, 5 aa, Artificial sequence
  antibody binding epitop of TNF-related apoptosis-inducing ligand (TRAIL)
LGRKI

SEQ ID NO: 50 - protein, 5 aa, Artificial sequence
  antibody binding epitop of TNF-related apoptosis-inducing ligand (TRAIL)
GRKIN

SEQ ID NO: 51 - protein, 6 aa, Artificial sequence
  antibody binding epitop of TNF-related apoptosis-inducing ligand (TRAIL)
RGRSNT

SEQ ID NO: 52 - protein, 17 aa, Artificial sequence
  antibody binding epitop of TNF-related apoptosis-inducing ligand (TRAIL)
ITGTRGRSNTLSSPNSK

SEQ ID NO: 53 - protein, 6 aa, Artificial sequence
  antibody binding epitop of TNF-related apoptosis-inducing ligand (TRAIL)
RSNTLS

SEQ ID NO: 54 - protein, 6 aa, Artificial sequence
  antibody binding epitop of TNF-related apoptosis-inducing ligand (TRAIL)
SNTLSS

SEQ ID NO: 55 - protein, 6 aa, Artificial sequence
  antibody binding epitop of TNF-related apoptosis-inducing ligand (TRAIL)
NTLSSP

SEQ ID NO: 56 - protein, 6 aa, Artificial sequence
  antibody binding epitop of TNF-related apoptosis-inducing ligand (TRAIL)
TLSSPN

SEQ ID NO: 57 - protein, 6 aa, Artificial sequence
  antibody binding epitop of TNF-related apoptosis-inducing ligand (TRAIL)
LSSPNS

SEQ ID NO: 58 - protein, 6 aa, Artificial sequence
  antibody binding epitop of TNF-related apoptosis-inducing ligand (TRAIL)
SSPNSK

SEQ ID NO: 59 - protein, 6 aa, Artificial sequence
  antibody binding epitop of TNF-related apoptosis-inducing ligand (TRAIL)
SPNSKN

SEQ ID NO: 60 - protein, 6 aa, Artificial sequence
  antibody binding epitop of TNF-related apoptosis-inducing ligand (TRAIL)
PNSKNE

SEQ ID NO: 61 - protein, 6 aa, Artificial sequence
  antibody binding epitop of TNF-related apoptosis-inducing ligand (TRAIL)
NSKNEK

SEQ ID NO: 62 - protein, 6 aa, Artificial sequence
  antibody binding epitop of TNF-related apoptosis-inducing ligand (TRAIL)
SKNEKA

SEQ ID NO: 63 - protein, 6 aa, Artificial sequence
  antibody binding epitop of TNF-related apoptosis-inducing ligand (TRAIL)
KNEKAL

SEQ ID NO: 64 - protein, 6 aa, Artificial sequence
  antibody binding epitop of TNF-related apoptosis-inducing ligand (TRAIL)
NEKALG

SEQ ID NO: 65 - protein, 6 aa, Artificial sequence
  antibody binding epitop of TNF-related apoptosis-inducing ligand (TRAIL)
EKALGR

SEQ ID NO: 66 - protein, 6 aa, Artificial sequence
  antibody binding epitop of TNF-related apoptosis-inducing ligand (TRAIL)
KALGRK

SEQ ID NO: 67 - protein, 6 aa, Artificial sequence
  antibody binding epitop of TNF-related apoptosis-inducing ligand (TRAIL)
ALGRKI

SEQ ID NO: 68 - protein, 6 aa, Artificial sequence
  antibody binding epitop of TNF-related apoptosis-inducing ligand (TRAIL)
LGRKIN

SEQ ID NO: 69 - protein, 4 aa, Artificial sequence
  antibody binding epitop of TNF-related apoptosis-inducing ligand (TRAIL)
EEIK

SEQ ID NO: 70 - protein, 4 aa, Artificial sequence
  antibody binding epitop of TNF-related apoptosis-inducing ligand (TRAIL)
EIKE

SEQ ID NO: 71 - protein, 4 aa, Artificial sequence
  antibody binding epitop of TNF-related apoptosis-inducing ligand (TRAIL)
IKEN

SEQ ID NO: 72 - protein, 4 aa, Artificial sequence
  antibody binding epitop of TNF-related apoptosis-inducing ligand (TRAIL)
KENT

SEQ ID NO: 73 - protein, 4 aa, Artificial sequence
  antibody binding epitop of TNF-related apoptosis-inducing ligand (TRAIL)
ENTK

SEQ ID NO: 74 - protein, 4 aa, Artificial sequence
  antibody binding epitop of TNF-related apoptosis-inducing ligand (TRAIL)
NTKN

SEQ ID NO: 75 - protein, 4 aa, Artificial sequence
  antibody binding epitop of TNF-related apoptosis-inducing ligand (TRAIL)
TKND

SEQ ID NO: 76 - protein, 4 aa, Artificial sequence
  antibody binding epitop of TNF-related apoptosis-inducing ligand (TRAIL)
KNDK

SEQ ID NO: 77 - protein, 4 aa, Artificial sequence
  antibody binding epitop of TNF-related apoptosis-inducing ligand (TRAIL)
NDKQ

SEQ ID NO: 78 - protein, 4 aa, Artificial sequence
  antibody binding epitop of TNF-related apoptosis-inducing ligand (TRAIL)
DKQM

SEQ ID NO: 79 - protein, 5 aa, Artificial sequence
  antibody binding epitop of TNF-related apoptosis-inducing ligand (TRAIL)
EEIKE

SEQ ID NO: 80 - protein, 5 aa, Artificial sequence
  antibody binding epitop of TNF-related apoptosis-inducing ligand (TRAIL)
EIKEN

SEQ ID NO: 81 - protein, 5 aa, Artificial sequence
  antibody binding epitop of TNF-related apoptosis-inducing ligand (TRAIL)
IKENT

SEQ ID NO: 82 - protein, 5 aa, Artificial sequence
  antibody binding epitop of TNF-related apoptosis-inducing ligand (TRAIL)
KENTK

SEQ ID NO: 83 - protein, 5 aa, Artificial sequence
  antibody binding epitop of TNF-related apoptosis-inducing ligand (TRAIL)
ENTKN

SEQ ID NO: 84 - protein, 5 aa, Artificial sequence
  antibody binding epitop of TNF-related apoptosis-inducing ligand (TRAIL)
NTKND

SEQ ID NO: 85 - protein, 6 aa, Artificial sequence
  antibody binding epitop of TNF-related apoptosis-inducing ligand (TRAIL)
NTKNDK

SEQ ID NO: 86 - protein, 6 aa, Artificial sequence
  antibody binding epitop of TNF-related apoptosis-inducing ligand (TRAIL)
TKNDKQ

SEQ ID NO: 87 - protein, 6 aa, Artificial sequence
  antibody binding epitop of TNF-related apoptosis-inducing ligand (TRAIL)
KNDKQM

SEQ ID NO: 88 - protein, 6 aa, Artificial sequence
  antibody binding epitop of TNF-related apoptosis-inducing ligand (TRAIL)
EEIKEN

SEQ ID NO: 89 - protein, 6 aa, Artificial sequence
  antibody binding epitop of TNF-related apoptosis-inducing ligand (TRAIL)
EIKENT

SEQ ID NO: 90 - protein, 6 aa, Artificial sequence
  antibody binding epitop of TNF-related apoptosis-inducing ligand (TRAIL)
IKENTK

SEQ ID NO: 91 - protein, 6 aa, Artificial sequence
  antibody binding epitop of TNF-related apoptosis-inducing ligand (TRAIL)
KENTKN

SEQ ID NO: 92 - protein, 6 aa, Artificial sequence
  antibody binding epitop of TNF-related apoptosis-inducing ligand (TRAIL)
ENTKND

SEQ ID NO: 93 - protein, 6 aa, Artificial sequence
  antibody binding epitop of TNF-related apoptosis-inducing ligand (TRAIL)
NTKNDK

SEQ ID NO: 94 - protein, 6 aa, Artificial sequence
  antibody binding epitop of TNF-related apoptosis-inducing ligand (TRAIL)
TKNDKQ

SEQ ID NO: 95 - protein, 6 aa, Artificial sequence
  antibody binding epitop of TNF-related apoptosis-inducing ligand (TRAIL)
KNDKQM

SEQ ID NO: 96 - protein, 281 aa, homo sapiens [sequence omitted]

SEQ ID NO: 97 - protein, 11 aa, Artificial sequence
  consensus motif of Mono 2
ITGTRGRSNTL

SEQ ID NO: 98 - protein, 7 aa, Artificial sequence
  consensus motif of Mono 26
KENTKND

SEQ ID NO: 99 - protein, 7 aa, Artificial sequence
  linker at the C- and N-terminus of TRAIL to avoid truncated peptides.
GSGSGSG

SEQ ID NO: 100 - protein, 10 aa, Artificial sequence
  HA control peptide
YPYDVPDYAG

SEQ ID NO: 101 - protein, 9 aa, Artificial sequence
  antibody binding epitop of TNF-related apoptosis-inducing ligand (TRAIL)
ETISTVQEK

SEQ ID NO: 102 - protein, 8 aa, Artificial sequence
  antibody binding epitop of TNF-related apoptosis-inducing ligand (TRAIL)
NISPLVRE

SEQ ID NO: 103 - protein, 6 aa, Artificial sequence
  antibody binding epitop of TNF-related apoptosis-inducing ligand (TRAIL)
ERGPQR

SEQ ID NO: 104 - protein, 8 aa, Artificial sequence
  antibody binding epitop of TNF-related apoptosis-inducing ligand (TRAIL)
RGRSNTLS

SEQ ID NO: 105 - protein, 7 aa, Artificial sequence
  antibody binding epitop of TNF-related apoptosis-inducing ligand (TRAIL)
LSSPNSK

SEQ ID NO: 106 - protein, 8 aa, Artificial sequence
  antibody binding epitop of TNF-related apoptosis-inducing ligand (TRAIL)
KALGRKIN

SEQ ID NO: 107 - protein, 7 aa, Artificial sequence
  antibody binding epitop of TNF-related apoptosis-inducing ligand (TRAIL)
FRFQEEI

SEQ ID NO: 108 - protein, 6 aa, Artificial sequence
  antibody binding epitop of TNF-related apoptosis-inducing ligand (TRAIL)
IKENTK

SEQ ID NO: 109 - protein, 8 aa, Artificial sequence
  antibody binding epitop of TNF-related apoptosis-inducing ligand (TRAIL)
SVTNEHLI

SEQ ID NO: 110 - protein, 13 aa, Artificial sequence
  antibody binding epitop of TNF-related apoptosis-inducing ligand (TRAIL)
MKSARNSCWSKDA

SEQ ID NO: 111 - protein, 36 aa, Artificial sequence
  antibody binding epitop of TNF-related apoptosis-inducing ligand (TRAIL)
GLYSIYQGGIFELKENDRIFVSVTNEHLIDMDHEAS

SEQ ID NO: 112 - protein, 12 aa, Artificial sequence
  antibody binding epitop of TNF-related apoptosis-inducing ligand (TRAIL)
SNTLSSPNSKNE

SEQ ID NO: 113 - protein, 19 aa, Artificial sequence
  antibody binding epitop of TNF-related apoptosis-inducing ligand (TRAIL)
VLIVIFTVLLQSLCVAVTY

SEQ ID NO: 114 - protein, 18 aa, Artificial sequence
  antibody binding epitop of TNF-related apoptosis-inducing ligand (TRAIL)
CTNEHLIDMDHEASFFGA

(19) United States
(12) Patent Application Publication
(10) Pub. No.: US 2020/0325219 A1
(43) Pub. Date: Oct. 15, 2020

(54) ANTI-TRAIL ANTIBODIES AND METHODS OF USE

(71) Applicant: MeMed Diagnostics Ltd., Tirat HaCarmel (IL)

(72) Inventors: Olga BOICO, Haifa (IL); Salit TZABAN, Nofit (IL); Kfir OVED, Hof HaCarmel (IL); Assaf COHEN-DOTAN, Natania (IL); Eran EDEN, Haifa (IL)

(73) Assignee: MeMed Diagnostics Ltd., Tirat HaCarmel (IL)

(21) Appl. No.: 16/868,616

(22) Filed: May 7, 2020

Related U.S. Application Data
(63) Continuation of application No. PCT/IL2018/051185, filed on Nov. 6, 2018.
(60) Provisional application No. 62/582,385, filed on Nov. 7, 2017.

(51) Int. Cl. C07K 16/24; G01N 33/543; G01N 33/577

(52) U.S. Cl. CPC C07K 16/241; C07K 2317/92; G01N 33/577; G01N 33/543

(57) ABSTRACT

An antibody comprising an antigen recognition domain that binds specifically the extracellular domain of TNF-related apoptosis-inducing ligand (TRAIL) between amino acids 95-155 and/or amino acids 190-210 is disclosed. Uses thereof are also disclosed.

### RELATED APPLICATIONS

This application is a US Continuation of PCT Patent Application No. PCT/IL2018/051185 having international filing date of Nov. 6, 2018 which claims the benefit of priority under 35 USC § 119(e) of U.S. Provisional Patent Application No. 62/582,385 filed on Nov. 7, 2017. The contents of the above applications are all incorporated by reference as if fully set forth herein in their entirety.

### SEQUENCE LISTING STATEMENT

The ASCII file, entitled 82193SequenceListing.txt, created on May 7, 2020, comprising 29,002 bytes, submitted concurrently with the filing of this application is incorporated herein by reference.

### FIELD AND BACKGROUND OF THE INVENTION

The present invention, in some embodiments thereof, relates to TRAIL antibodies having high specificity and uses thereof.

Antibiotics (Abx) are the world's most prescribed class of drugs with a 25-30 billion $US global market. Abx are also the world's most misused drug with a significant fraction of all drugs (40-70%) being wrongly prescribed (Linder, J. A. and R. S. Stafford 2001; Scott, J. G. and D. Cohen, et al. 2001; Davey, P. and E. Brown, et al. 2006; Cadieux, G. and R. Tamblyn, et al. 2007; Pulcini, C. and E. Cua, et al. 2007), (“CDC—Get Smart: Fast Facts About Antibiotic Resistance” 2011).

One type of Abx misuse is when the drug is administered in case of a non-bacterial disease, such as a viral infection, for which Abx is ineffective. For example, according to the USA center for disease control and prevention CDC, over 60 Million wrong Abx prescriptions are given annually to treat flu in the US. The health-care and economic consequences of Abx over-prescription include: (i) the cost of antibiotics that are unnecessarily prescribed globally, estimated at >$10 billion annually; (ii) side effects resulting from unnecessary Abx treatment are reducing quality of healthcare, causing complications and prolonged hospitalization (e.g. allergic reactions, Abx associated diarrhea, intestinal yeast etc.) and (iii) the emergence of resistant strains of bacteria as a result of the overuse (the CDC has declared the rise in antibiotic resistance of bacteria as “one of the world's most pressing health problems in the 21^{st} century” (Arias, C. A. and B. E. Murray 2009; “CDC—About Antimicrobial Resistance” 2011).

Antibiotics under-prescription is not uncommon either. For example up to 15% of adult bacterial pneumonia hospitalized patients in the US receive delayed or no Abx treatment, even though in these instances early treatment can save lives and reduce complications (Houck, P. M. and D. W. Bratzler, et al. 2002).

Technologies for infectious disease diagnosis have the potential to reduce the associated health and financial burden associated with Abx misuse. Ideally, such a technology should: (i) accurately differentiate between a bacterial and viral infections; (ii) be rapid (within minutes); (iii) be able to differentiate between pathogenic and non-pathogenic bacteria that are part of the body's natural flora; (iv) differentiate between mixed co-infections and pure viral infections and (v) be applicable in cases where the pathogen is inaccessible (e.g. sinusitis, pneumonia, otitis-media, bronchitis, etc).

Current solutions (such as culture, PCR and immunoassays) do not fulfill all these requirements: (i) Some of the assays yield poor diagnostic accuracy (e.g. low sensitivity or specificity)(Uyeki et al. 2009), and are restricted to a limited set of bacterial or viral strains; (ii) they often require hours to days; (iii) they do not distinguish between pathogenic and non-pathogenic bacteria (Del Mar, C 1992), thus leading to false positives; (iv) they often fail to distinguish between a mixed and a pure viral infections and (v) they require direct sampling of the infection site in which traces of the disease causing agent are searched for, thus prohibiting the diagnosis in cases where the pathogen resides in an inaccessible tissue, which is often the case.

Consequentially, there still a diagnostic gap, which in turn often leads physicians to either over-prescribe Abx (the “Just-in-case-approach”), or under-prescribe Abx (the “Wait-and-see-approach”) (Little, P. S. and I. Williamson 1994; Little, P. 2005; Spiro, D. M. and K. Y. Tay, et al. 2006), both of which have far reaching health and financial consequences.

TNF-related apoptosis-inducing ligand (TRAIL) is a member of the tumor necrosis factor family implicated in programmed cell death. It has been demonstrated that TRAIL can serve as a useful biomarker for distinguishing between bacterial and viral infections (WO 2013/117746).

### SUMMARY OF THE INVENTION

According to an aspect of some embodiments of the present invention there is provided an antibody comprising an antigen recognition domain that binds specifically the extracellular domain of TNF-related apoptosis-inducing ligand (TRAIL) between amino acids 95-155 and/or amino acids 190-210, with the proviso that the antibody is not an antibody set forth in Table 2.

According to an aspect of some embodiments of the present invention there is provided an antibody comprising an antigen recognition domain that binds specifically to at least one epitope of TNF-related apoptosis-inducing ligand (TRAIL), wherein the at least one epitope is in an amino acid sequence selected from the group consisting of SEQ ID NOs: 1-95, 97, 98 and 101-109, with the proviso that the antibody is not an antibody set forth in Table 2.

According to an aspect of some embodiments of the present invention there is provided a kit comprising at least two antibodies, each of the two antibodies comprising an antigen recognition domain that binds specifically the extracellular domain of TNF-related apoptosis-inducing ligand (TRAIL), wherein each of the at least two antibodies bind to a non-identical epitope which is in an amino acid sequence selected from the group consisting of SEQ ID NO: 1-95, 97, 98 and 101-109.

According to an aspect of some embodiments of the present invention there is provided an isolated polynucleotide comprising a nucleic acid sequence encoding the antibody described herein.

According to an aspect of some embodiments of the present invention there is provided an expression vector comprising the polynucleotide described herein, operably linked to a cis-acting regulatory element.

According to an aspect of some embodiments of the present invention there is provided a cell comprising the polynucleotide described herein or the expression vector described herein.

According to an aspect of some embodiments of the present invention there is provided a method of diagnosing an infectious disease in a subject in need thereof comprising determining the amount of TRAIL in a blood sample of the subject, the determining is effected using at least one antibody which comprises an antigen recognition domain that binds specifically to at least one epitope of TNF-related apoptosis-inducing ligand (TRAIL), wherein the at least one epitope is in an amino acid sequence selected from the group consisting of SEQ ID NOs: 1-95, 97, 98 and 101-109.

According to an aspect of some embodiments of the present invention there is provided a method of generating a TRAIL antibody comprising immunizing a non-human animal with a peptide having an amino acid sequence selected from the group consisting of SEQ ID NO: 1-95, 97, 98 and 101-109, wherein the peptide is no longer than 20 amino acids, thereby generating the TRAIL antibody.

According to an aspect of some embodiments of the present invention there is provided a method of generating a TRAIL antibody comprising expressing the polynucleotide described herein in a cell, thereby generating the antibody.

According to an aspect of some embodiments of the present invention there is provided a composition of matter comprising a carrier and a peptide which is no longer than 20 amino acids, wherein the peptide comprises an amino acid sequence selected from the group consisting of SEQ ID NO: 1-95, 97, 98 and 101-109, the carrier being selected from the group consisting of bovine serum albumin (BSA), keyhole limpet hemocyanin (KLH), ovalbumin (OVA) and poly-Lys.

According to an aspect of some embodiments of the present invention there is provided a method of detecting TRAIL in a biological sample comprising:

(a) contacting the sample with the antibody described herein; and

(b) detecting the antibody.

According to an aspect of some embodiments of the present invention there is provided a composition of matter comprising blood and the antibody described herein.

According to some embodiments of the invention, the antigen recognition domain binds the TRAIL between amino acids 130-140 and/or 195-205.

According to some embodiments of the invention, the antigen recognition domain binds to at least one epitope which is in an amino acid sequence selected from the group consisting of SEQ ID NO: 1-8, 10-95 and 101-108.

According to some embodiments of the invention, the antigen recognition domain binds to at least four contiguous amino acids of SEQ ID NO: 15 (FRFQEEIKENTKND) or SEQ ID NO: 52 (ITGTRGRSNTLSSPNSK).

According to some embodiments of the invention, the antibody is a monoclonal antibody.

According to some embodiments of the invention, the at least one epitope is in the amino acid sequence as set forth in SEQ ID NO: 4 or 74.

According to some embodiments of the invention, the antibody is a polyclonal antibody which binds to at least two epitopes of the TRAIL, wherein the at least two epitopes are in an amino acid sequence selected from the group consisting of SEQ ID NOs: 1-95, 97, 98 and 101-109.

According to some embodiments of the invention, the antibody binds to at least three epitopes of the TRAIL.

According to some embodiments of the invention, the antibody binds to each of the epitopes in the sequences set forth in SEQ ID NOs: 1-9.

According to some embodiments of the invention, the antibody is capable of binding the TRAIL when presented in a blood sample at a concentration below 70 pg/ml, as determined by an immunoassay.

According to some embodiments of the invention, the blood sample comprises whole blood.

According to some embodiments of the invention, the blood sample comprises a fraction of whole blood.

According to some embodiments of the invention, the antibody is of an IgG subclass.

According to some embodiments of the invention, the antibody comprises a detectable moiety.

According to some embodiments of the invention, the antibody is soluble.

According to some embodiments of the invention, the antibody is attached to a solid support.

According to some embodiments of the invention, the antibody is a recombinant antibody.

According to some embodiments of the invention, at least one of the at least two antibodies is a monoclonal antibody.

According to some embodiments of the invention, the diagnosing comprises distinguishing between a bacterial infection and a viral infection.

According to some embodiments of the invention, when the amount of TRAIL is below a predetermined value, a bacterial infection is ruled in.

According to some embodiments of the invention, when the amount of TRAIL is above a predetermined value, a viral infection is ruled in.

According to some embodiments of the invention, the diagnosing comprises distinguishing between a mixed bacterial/viral infection and a viral infection or a mixed bacterial/viral infection and a bacterial infection.

According to some embodiments of the invention, the diagnosing comprises determining the severity of the disease.

According to some embodiments of the invention, the disease is selected from the group consisting of meningitis, sepsis, pneumonia, septic arthritis and cellulitis, bacteremia, urinary tract infection (UTI), Pyelonephritis, Meningococcal Disease, Invasive; Staphylococcus Aureus Infections; Drug resistant (MRSA, VISA, VRSA) Streptococcal Disease, Group A Invasive or Streptococcal TSS and Streptococcal Disease.

According to some embodiments of the invention, the determining is effected by lateral flow immunoassay, flow cytometry, radioimmunoassay, immunofluorescence or by an enzyme-linked immunosorbent assay.

According to some embodiments of the invention, the method further comprises determining the amount of a determinant selected from the group consisting of CRP, IP10, IL-6, NGAL, PCT and MX1.

According to some embodiments of the invention, the method further comprises isolating the TRAIL antibody.

According to some embodiments of the invention, the method further comprises analyzing the affinity of the TRAIL antibody to TRAIL.

According to some embodiments of the invention, the method further comprises isolating the antibody.

According to some embodiments of the invention, the antibody is a monoclonal antibody.

According to some embodiments of the invention, the detecting is effected using a second antibody.

According to some embodiments of the invention, the second antibody binds to the TRAIL at a different epitope to the antibody used in step (a).

According to some embodiments of the invention, the second antibody binds to the TRAIL at an epitope that overlaps the epitope of the antibody used in step (a).

According to some embodiments of the invention, the monoclonal antibody is attached to a solid support.

According to some embodiments of the invention, the second antibody is an antibody which is described herein.

According to some embodiments of the invention, the second antibody is a polyclonal antibody.

According to some embodiments of the invention, the detecting is effected by lateral flow immunoassay, flow cytometry, radioimmunoassay, immunofluorescence or by an enzyme-linked immunosorbent assay.

According to some embodiments of the invention, the biological sample is a blood sample.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: Cytoplasmic, transmembrane, and extracellular domains of the TRAIL protein marked in yellow, gray, and light blue (respectively) on TRAIL amino acid sequence (SEQ ID NO: 96).

FIGS. 2A-B: (A) Read outs at scanning intensities of 6/7 (red/green) of goat antibody Poly at a concentration of 1 μg/ml after staining with secondary and control antibodies. (B) Multiple epitope spot patterns identified by elevated florescence levels, as measured on goat antibody Poly at a concentration of 1 μg/ml.

FIGS. 3A-B: (A) Read outs at scanning intensity of 7 (red) of mouse antibody Mono 2 at concentration of 250 μg/ml after staining with secondary and control antibodies. (B) Three epitope spot patterns identified with all three peptide lengths with the consensus motif ITGTRGRSNTL (SEQ ID NO: 97), identified by elevated florescence levels, as measured on mouse antibody Mono 2 at concentrations of 10 μg/ml, 100 μg/ml and 250 μg/ml.

FIGS. 4A-B: (A) Read outs at scanning intensity of 7 (red) of on mouse antibody Mono 26 at concentration of 10 250 μg/ml after staining with secondary goat anti-mouse IgG (H+L) DyLight680 antibody. (B) Moderate monoclonal antibody response against clear epitope spot patterns formed by adjacent peptides with the consensus motif KENTKND (SEQ ID NO: 98) with all three peptide lengths, identified by elevated florescence levels, as measured on mouse antibody Mono 26 at concentrations of 10 μg/ml, 100 μg/ml and 250 μg/ml.

FIG. 5: Identified epitopes of Poly, Mono 2 and Mono26 antibodies on the TRAIL amino acid sequence, marked by red and green rectangles respectively (SEQ ID NO: 96).

FIGS. 6A-B: (A) Exemplary data demonstrating detection of TRAIL protein in different concentrations using the Mono 2 (first capture) and Polyclonal antibody (second detection) using recombinant TRAIL protein at known concentrations. (B) Exemplary data demonstrating detection of TRAIL protein in clinical blood samples measured once using the Mono 2 (first capture) and Polyclonal antibody (second detection; Y-axis), and once using a commercial ELISA kit (MeMed, IL; X-axis). High correlation is observed between the two detection methods (R²=0.9844).

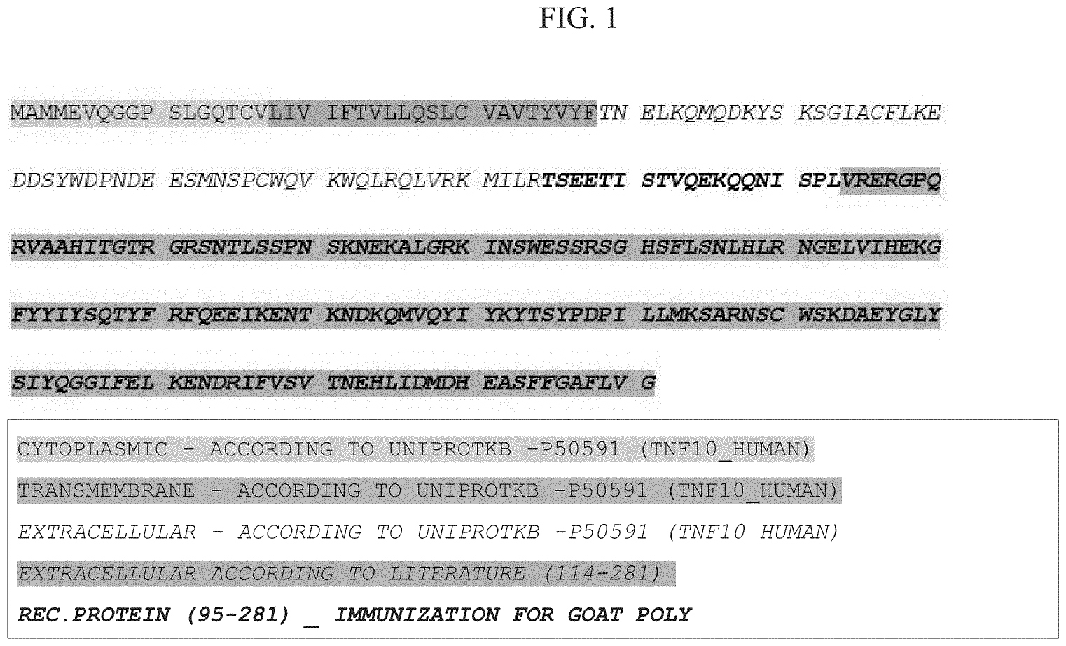

### DESCRIPTION OF SPECIFIC EMBODIMENTS OF THE INVENTION

The present invention, in some embodiments thereof, relates to TRAIL antibodies having high specificity and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

TNF-related apoptosis-inducing ligand (TRAIL) is a member of the tumor necrosis factor family implicated in programmed cell death. It has been demonstrated that TRAIL can serve as a useful biomarker for distinguishing between bacterial and viral infections (WO 2013/117746).

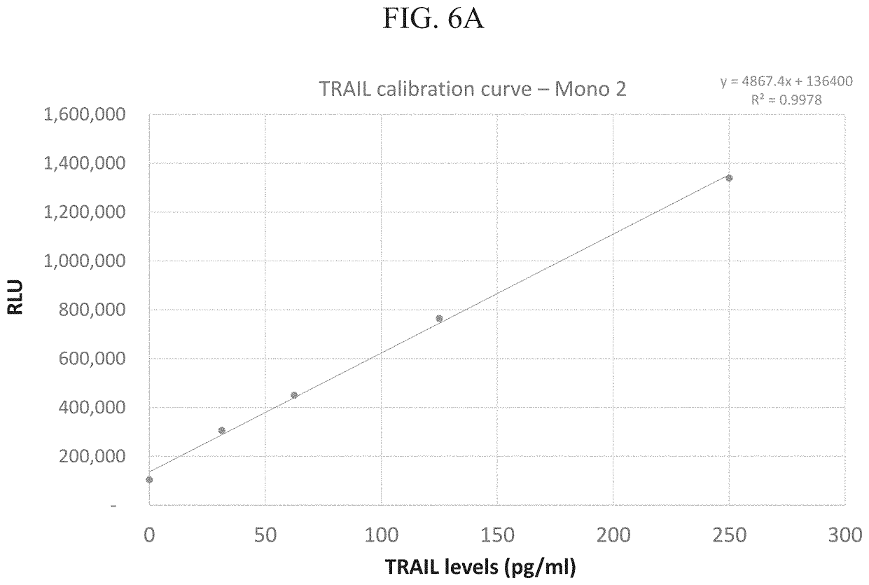

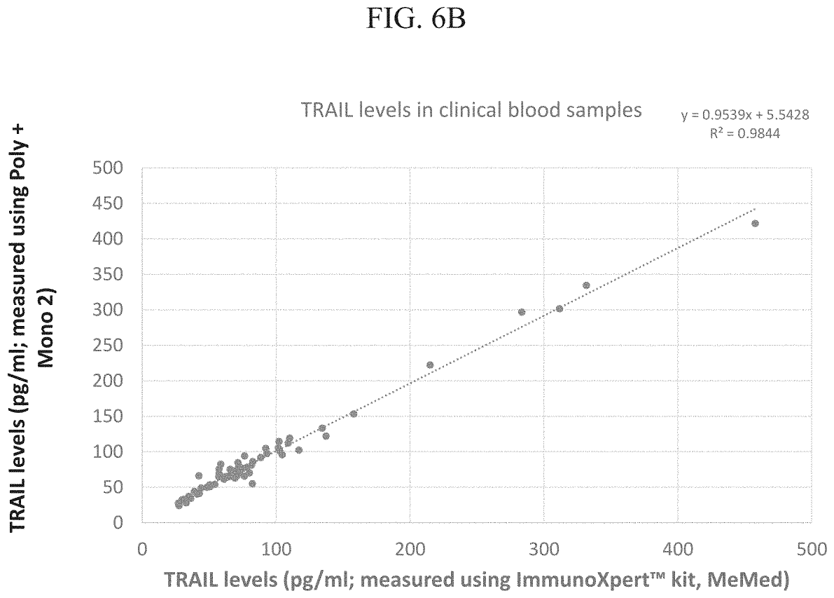

Whilst searching for TRAIL antibodies that can be used for diagnostic methods, the present inventors identified a polyclonal antibody with very high specificity and affinity to human TRAIL (R&D Systems AF375). Specifically, this polyclonal antibody was found to be useful for detecting very low levels of TRAIL in blood samples such as those derived from bacterially infected patients (see FIG. 6B).

Through laborious experimentation, the present inventors identified the epitopes on the TRAIL protein to which the polyclonal antibody binds. In addition, the present inventors performed epitope mapping on a monoclonal antibody (referred to herein as Mono 2) that also shows very high specificity and affinity for TRAIL protein in blood samples (see FIG. 6A).

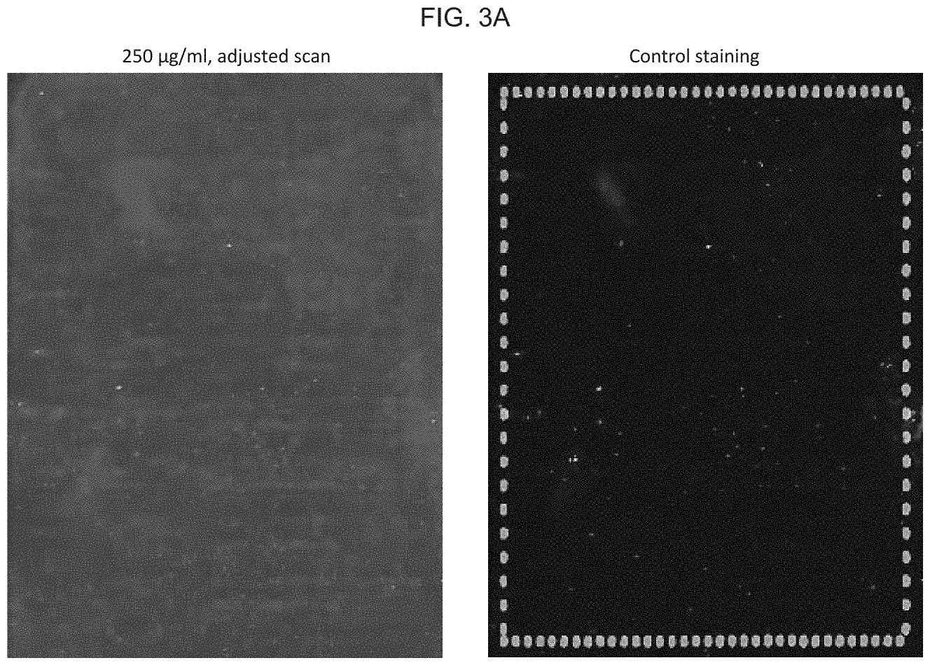

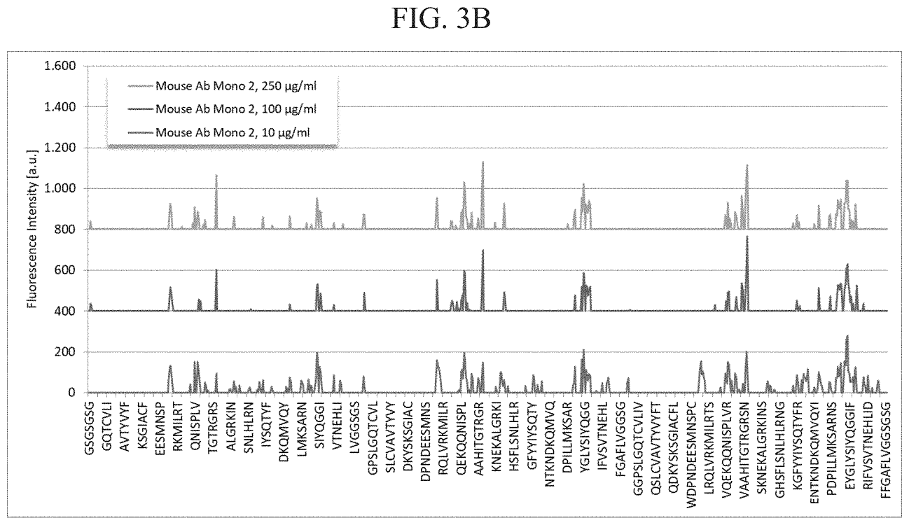

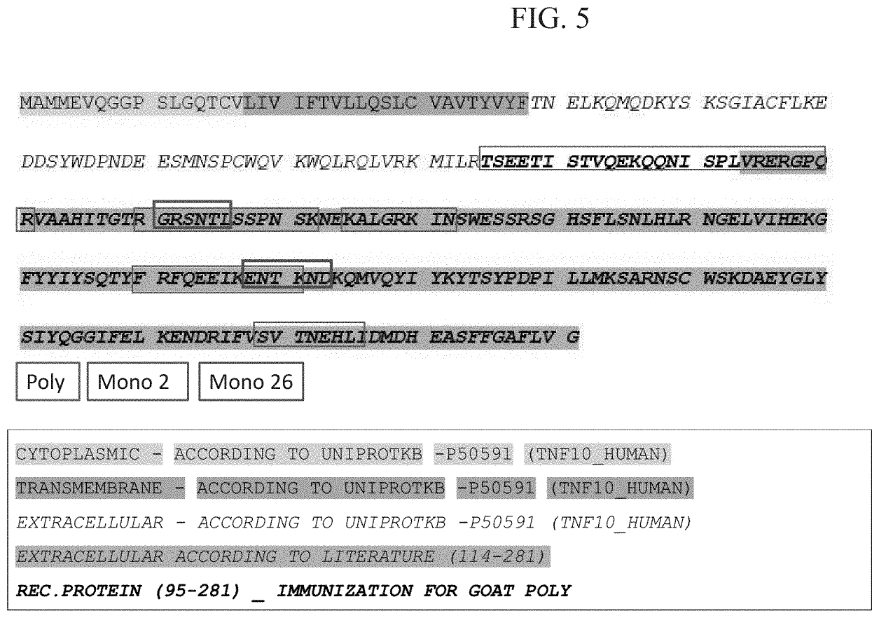

Surprisingly, the present inventors found that the monoclonal antibody and the polyclonal antibody bound to overlapping sites (SEQ ID NO: 4)—FIGS. 3A-B and FIG. 5.

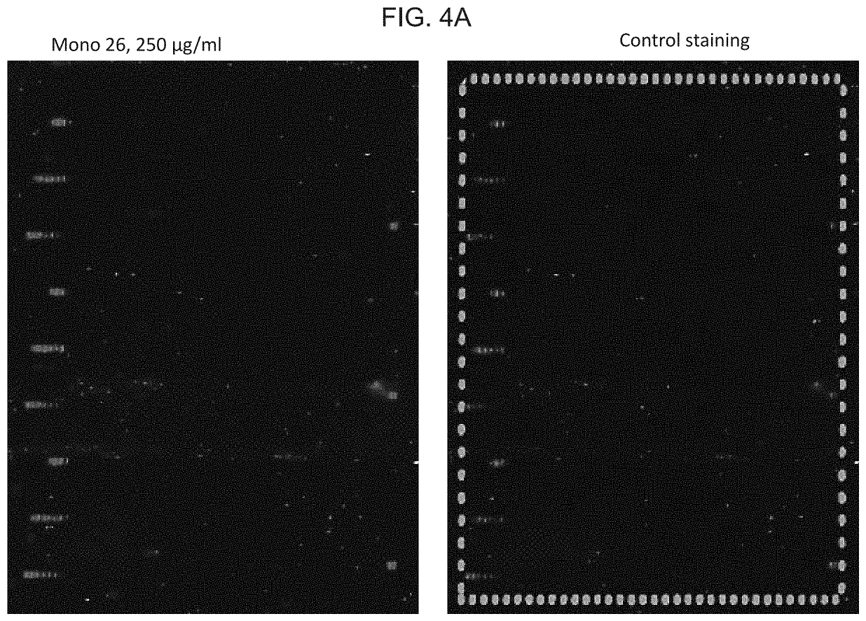

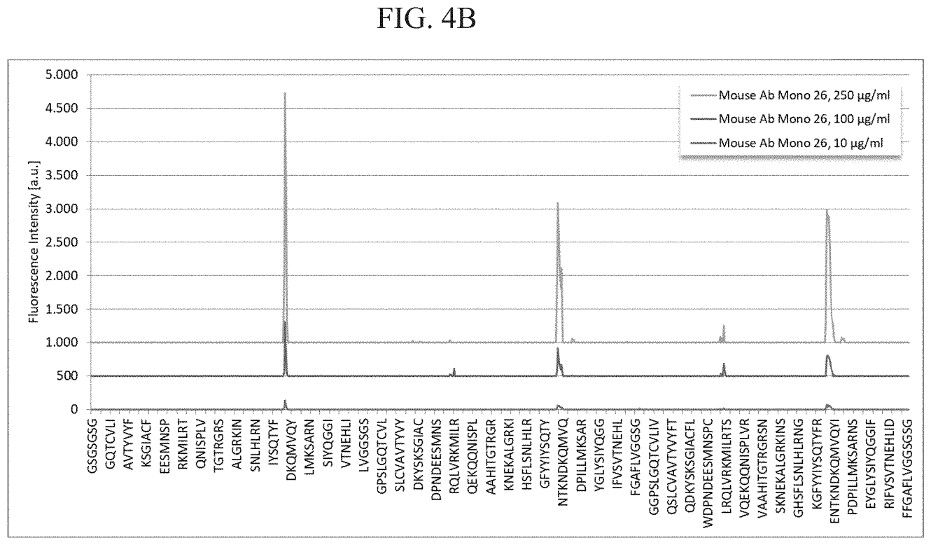

Encouraged by these results, the present inventors analyzed a novel monoclonal antibody (referred to herein as Mono 26) which binds to TRAIL in blood samples. Epitope mapping showed that the novel monoclonal antibody too bound to an overlapping site to the polyclonal antibody—SEQ ID NO: 74 (FIGS. 4A-B and FIG. 5).

Thus, the present inventors conclude that the polyclonal epitopes uncovered by the present screening assay are of functional importance and accordingly any antibody (whether monoclonal or polyclonal) that binds to at least one of the discovered epitopes will be useful for detecting TRAIL in general and in blood samples in particular.

Thus, according to a first aspect of the present invention there is provided an antibody comprising an antigen recognition domain that binds specifically to the extracellular domain of TNF-related apoptosis-inducing ligand (TRAIL) between amino acids 95-155 and/or amino acids 190-210, with the proviso that the antibody is not an antibody set forth in Table 2.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen in an MHC restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989), a compromise between Kabat and Chothia using Oxford Molecular's AbM antibody modeling software (now Accelrys™, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77].

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibodies may be generated via any one of several methods known in the art. In one embodiment, the generating comprises in vivo production of antibody molecules. In this embodiment, a non-human animal is injected with a peptide comprising at least one of the epitopes described herein. The peptide may be between 4-50 amino acids, 4-40 amino acids, 4-30 amino acids or 4-20 amino acids.

In cases where the invention compounds are too small to elicit a strong immunogenic response, such antigens (haptens) can be coupled to antigenically neutral carriers such as keyhole limpet hemocyanin (KLH) or serum albumin [e.g., bovine serum albumin (BSA)] carriers (see U.S. Pat. Nos. 5,189,178 and 5,239,078). Coupling to carrier can be effected using methods well known in the art; For example, direct coupling to amino groups can be effected and optionally followed by reduction of imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art. The antisera obtained can be used directly or monoclonal antibodies may be obtained as described hereinabove.

Other methods for generating antibodies include screening immunoglobulin libraries or panels of highly specific binding reagents as disclosed [Orlandi D. R. et al. (1989) Proc. Natl. Acad. Sci. 86:3833-3837, Winter G. et al. (1991) Nature 349:293-299] or generation of monoclonal antibody molecules by continuous cell lines in culture. These include but are not limited to, the hybridoma technique, the human B-cell hybridoma technique, and the Epstein-Bar-Virus (EBV)-hybridoma technique [Kohler G., et al. (1975) Nature 256:495-497, Kozbor D., et al. (1985) J. Immunol. Methods 81:31-42, Cote R. J. et al. (1983) Proc. Natl. Acad. Sci. 80:2026-2030, Cole S. P. et al. (1984) Mol. Cell. Biol. 62:109-120].

In addition, the antibody may be prepared using recombinant means as further described herein below.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

In an embodiment in which the antibody is a full length antibody, the heavy and light chains of an antibody of the invention may be full-length (e.g., an antibody can include at least one, and preferably two, complete heavy chains, and at least one, or two, complete light chains) or may include an antigen-binding portion (a Fab, F(ab′).sub.2, Fv or a single chain Fv fragment (“scFv”)). In other embodiments, the antibody heavy chain constant region is chosen from, e.g., IgG1, IgG2, IgG3, IgG4, IgM, IgA1, IgA2, IgD, and IgE. In some embodiments, the immunoglobulin isotype is selected from IgG1, IgG2, IgG3, and IgG4, more particularly, IgG1 (e.g., human IgG1) or IgG4 (e.g., human IgG4).

TRAIL antibodies of the present invention may be produced using recombinant DNA technology.

Thus according to an aspect of the invention there is provided an isolated polynucleotide comprising a nucleic acid sequence encoding the antibody as described herein.

Also provided is an expression vector, comprising the polynucleotide operably linked to a cis-acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the antibody from a host cell in which it is placed. Preferably the signal sequence for this purpose is a mammalian signal sequence.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264, 166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein.

Such cells are typically selected for high expression of recombinant proteins (e.g., bacterial, plant or eukaryotic cells e.g., CHO, HEK-293 cells).

As mentioned, the antibody of this aspect of the present invention binds to the protein TRAIL.

As used herein “binding” or “binds” refers to an antibody-antigen mode of binding. In one embodiment, the antibody has a K_{D} below 100×10⁻⁶, as determined by Surface Plasmon Resonance assay (SPR), Biacore or any other assay.

As used herein the term “K_{D}” refers to the equilibrium dissociation constant between the antigen binding domain and its respective antigen.

Higher affinities are also contemplated e.g., 1-100×10⁻⁸ M, 1-100×10⁻⁹ M, 1-100×10⁻¹⁰ M, 1-100×10⁻¹¹ M or 1-100×10⁻¹² M.

TRAIL protein is a cytokine that belongs to the tumor necrosis factor (TNF) ligand family. Additional names of the gene encoding the protein include without limitations APO2L, TNF-related apoptosis-inducing ligand, TNFSF10 and CD253. This protein binds to several members of the TNF receptor superfamily such as TNFRSF10A/TRAILR1, TNFRSF10B/TRAILR2, TNFRSF10C/TRAILR3, TNFRSF10D/TRAILR4, and possibly also to TNFRSF11B/OPG. An exemplary amino acid sequence of TRAIL is set forth in SEQ ID NO: 96.

Additional information concerning TRAIL is provided in Table 1, herein below.

TABLE 1

| | | RefSeq DNA |
|---|---|---|
| Protein symbol | Full Gene Name | sequence |
| TRAIL | Tumor necrosis factor | NC_000003.12 |
| | superfamily member | NC_018914.2 |
| | 10 | NT_005612.17 |

Human TRAIL has an extracellular domain from amino acid 39-281 of the protein, wherein the numbering of the TRAIL protein is according to SEQ ID NO: 96.

As mentioned, the antibody of this aspect of the present invention binds the extracellular domain of TRAIL between amino acids 95-155 and/or amino acids 190-210.

According to a particular embodiment, the antibody binds (via the antigen recognition domain) to the extracellular domain of TRAIL between amino acids 130-140 and/or 195-205. Thus, the antigen recognition domain may bind to at least three, four, five, six, seven or more contiguous amino acids of SEQ ID NO: 12 or SEQ ID NO: 13 of the TRAIL protein—i.e. the epitope of the antibody lies in the sequence of SEQ ID NO:12 or 13.

Table 2 provides a list of antibodies that bind to TRAIL that are excluded from this aspect of the present invention.

TABLE 2
Table 2.

Mouse, Monoclonal (55B709-3) IgG (Thermo Fisher Scientific)
Rabbit, polyclonal (ab9959) unknown isotype (abeam)
Mouse, monoclonal (ab10516) IgG1, clone 75411.11 (abcam)
Rabbit, polyclonal (ab42121) IgG (abcam)
Rabbit, polyclonal (ab65121) IgG (abcam)
Rabbit, polyclonal (ab42243) IgG (abcam)
Rabbit, polyclonal (ab2056) IgG (abcam)
Mouse, monoclonal (biotin) (ab27322) IgG1 (abcam)
Mouse, monoclonal (ab183474) IgG1 (abcam)
Mouse, monoclonal (ab171261) IgG1, Clone: RIK-2 (abcam)
Rabbit, polyclonal (ab83147) IgG (abcam)
Chicken, polyclonal (ab26933) IgY (abcam)
Mouse, Monoclonal (11040-08) IgG2b (SouthernBiotech)
Rabbit, polyclonal (bs-1214R) IgG (Bioss Inc.)
Rabbit, polyclonal (bs-1214R-HRP) IgG (Bioss Inc.)
Rabbit, polyclonal (bs-1214R-Cy3, bs-1214R-Cy5, bs-1214R-Cy5.5, bs-1214R-
Cy7, bs-1214R-FITC) IgG, (Bioss Inc.)
Rabbit, polyclonal (bs-1214R-A488, bs-1214R-A555) IgG, (Bioss Inc.)
Mouse, Monoclonal (MAI-41027) IgG1 kappa, Clone 55B709.3 (Invitrogen
Antibodies)
Rabbit, Polyclonal (PA1-955, PA1-4172, PA1-4171, PA5-29625), IgG
(Invitrogen Antibodies)
Mouse, Mooclonal (MA5-23771) IgG1, clone: 124723 (Invitrogen Antibodies)
Mouse, Monoclonal (16-9927-82, 12-9927-42) IgG1 kappa, Clone RIK-2
(Invitrogen Antibodies)
Rabbit, polyclonal (200-401-H27) (Rockland Immunochemicals, Inc.)
Rabbit, Polyclonal (R32840), IgG, Immunogen: A recombinant protein
corresponding to amino acids N40-A272, NSJ Bioreagents
Rabbit, polyclonal (orbl63046, orbl93694, orbl26440) IgG, Immunogen:
Synthesized peptide derived from the Internal region of human TRAIL, Biorbyt
Rabbit, polyclonal (orb11509) IgG, Immunogen: synthetic peptide derived
between 195-271 amino acids of human Trail, Biorbyt
Rabbit, polyclonal (orb77097, orb77096, orb86822, orb96122,) IgG, Biorbyt
Mouse, Monoclonal (orb308373), IgG1, Clone: B-T24, Biorbyt
Mouse, Monoclonal (orb308369, orb308370, orb308371, orb308372), IgG1,
Clone: B-S23, Immunogen: Recombinant human TRAIL, Biorbyt
Rabbit, Polyclonal (orb311422), IgG, Biorbyt
Rabbit, Polyclonal (orb74315, orb225738), Biorbyt
Rabbit, Polyclonal (orb304632), IgG, Immunogen: synthetic peptide
encompassing a sequence within the center region of human CD253, Biorbyt
Rabbit, Polyclonal (orb227930, orb228996), IgG, Immunogen: A synthesized
peptide derived from human CD253, Biorbyt
Rabbit, Polyclonal (orb238273, orb238274, orb238275, orb238276), IgG,
Immunogen: Recombinant human Tumor necrosis factor ligand superfamily
member 10 protein, Biorbyt
Mouse, Monoclonal (orb303942), IgG, Immunogen: sTRAIL- highly pure
(>98%) recombinant human, Biorbyt
Rabbit, Polyclonal (orb303938, orb303941), IgG, Immunogen: sTRAIL- highly
pure (>98%) recombinant human, Biorbyt
Mouse, Monoclonal (308210, 308205, 308206, 308207, 308208, 308202,
308209) IgG1 kappa, Clone: RIK-2 (BioLegend)
Rabbit, Polyclonal (3045-100, 3045-30T) IgG (BioVision)
Rabbit Polyclonal (5354-100, 5354-30T) IgG (BioVision)
Rabbit, Polyclonal (GTX113043) IgG (GeneTex)
Rabbit, Polyclonal (GTX74230, GTX22435) IgG (GeneTex)
Mouse, Monoclonal (GTX12124) IgG1 (GeneTex)
Rabbit, Polyclonal (MBS840904, MBS 150492, MBS415624, MBS2537274,
MBS2539500, MBS7605156, MBS244256, MBS2002286, MBS2015805,
MBS715652, MBS715795, MBS715754, MBS127586, MBS551116,
MBS221503, MBS221579, MBS223155, MBS1750412, MBS460361,
MBS460360, MBS711224), IgG, (MyBioSource(dot)com)
Rabbit, Polyclonal (MBS8504081) (MyBioSource(dot)com)
Rabbit, Polyclonal (MBS852632, MBS9403450, MBS9408015, MBS691077,
MBS841388, MBS692252, MBS695591, MBS 1750412, MBS2032096),
(MyBioSource(dot)com)
Rabbit (MBS8501419, MBS8507780, MBS003932), (MyBioSource(dot)com)
Rabbit, Polyclonal (MBS854666), (MyBioSource(dot)com)
Mouse, Monoclonal (MBS690389), IgG1 kappa, (MyBioSource(dot)com)
Rabbit, Polyclonal (MBS565203), Recognizes soluble TRAIL,
(MyBioSource(dot)com)
Mouse, Monoclonal (MBS244256), IgG2b, (MyBioSource(dot)com)
Mouse, Monoclonal (MBS670246), IgG2b, clone: SB91c,
(MyBioSource(dot)com)
Mouse, Monoclonal (MBS670328), IgG2b, clone: SB91a,
(MyBioSource(dot)com)
Mouse, Monoclonal (MBS565040), IgG1, clone: RIK-2,
(MyBioSource(dot)com)
Mouse, Monoclonal (MBS2001279), IgG, (MyBioSource(dot)com)
Rabbit, Polyclonal (MBS8235785, MBS9412933, MBS853962), IgG,
Immunogen: Recognizes endogenous levels of CD253 protein,
(MyBioSource(dot)com)
Mouse, Monoclonal (MBS690229), IgG1, clone: #6D44,
(MyBioSource(dot)com)
Rabbit, (MBS8505410), IgG, (MyBioSource(dot)com)
Rabbit, Polyclonal (MBS9405266), detects endogenous level of total TNFSF10
antibody, (MyBioSource(dot)com)
Mouse, Monoclonal (MBS695019), sTRAIL, (MyBioSource(dot)com)
Rabbit, Polyclonal (MBS695365), sTRAIL, (MyBioSource(dot)com)
Mouse, Monoclonal (MBS219596), IgG1, clone: 200000,
(MyBioSource(dot)com)
Rabbit, polyclonal (70R-TR022) (Fitzgerald Industries International)
Rabbit, polyclonal (70R-TR029) IgG, Immunogen: raised in rabbit using
residues 223-235 [MKSARNSCWSKDA - SEQ ID NO: 110] of the C terminus
of the TRAIL protein as the immunogen (Fitzgerald Industries International)
Rat, Monoclonal (10R-6583, 61R-1383, 61R-1627), IgG2a kappa, clone: N2B2,
(Fitzgerald Industries International)
Mouse, Monoclonal (10R-T140A), IgG1 kappa, Immunogen: highly pure
recombinant human TRAIL was used, (Fitzgerald Industries International)
Rabbit, Polyclonal (70R-11579, 70R-12326, 70R-21430), IgG (Fitzgerald
Industries International)
Rabbit, Polyclonal (70R-13960), IgG, Immunogen: a synthetic peptide
corresponding to a sequence at the C-terminal of human TRAIL, (Fitzgerald
Industries International)
Rabbit, Polyclonal (70R-10455), Immunogen: the N terminal of TNFSF10
(Fitzgerald Industries International)
Rabbit Monoclonal (3219S) IgG (Cell Signaling Technology)
Goat Polyclonal (AF375) IgG (R&D Systems)
Mouse, Monoclonal, (MAB375-100, MAB375-SP, MAB375-500), IgG1, Clone
75411 (R&D Systems)
Mouse, Monoclonal (MAB3751-SP, MAB3751-100, MAB3751-500), IgG1,
Clone 124723 (R&D Systems)
Goat, Polyclonal (AF375-SP), IgG, (R&D Systems)
Mouse, Monoclonal (MAB687-SP, MAB687, FAB687A, FAB687P,
FAB687G), IgG1, Clone: 75402 (R&D Systems)
Goat, Polyclonal (BAF375), IgG, (R&D Systems)
Mouse, Monoclonal (NB100-56518, NB100-56518SS, NB100-56518B, NB100-
56518H, NB100-56518UV,) IgG1 Kappa, clone: 55B709.3, Immunogen- a
peptide corresponding to amino acids 17-35 of human TRAIL, (Novus
Biologicals)
Rabbit, Polyclonal (NBP2-23644, NBP2-23644SS, NBP1-77062), IgG,
Immunogen: peptide corresponding to 17 amino acids near the carboxy terminus
of human TRAIL. (Novus Biologicals)
Rabbit, Polyclonal (NBP2-24385), IgG, (Novus Biologicals)
Rabbit, Polyclonal (NB500-221), IgG, Immunogen: residues
[MKSARNSCWSKDA - SEQ ID NO: 110] of the C terminus of the TRAIL
protein (Novus Biologicals)
Rabbit, Polyclonal (NBP2-387440) IgG, Immunogen: a recombinant protein
corresponding to amino acids:
GLYSIYQGGIFELKENDRIFVSVTNEHLIDMDHEAS - SEQ ID NO: 111,
(Novus Biologicals)
Rabbit, polyclonal (NB500-220), IgG, Immunogen: residues
[SNTLSSPNSKNE - SEQ ID NO: 112] of the N terminus of the TRAIL protein
(Novus Biologicals)
Rabbit, Polyclonal (NBP2-20692), IgG, Immunogen: a sequence within the
center region of human TRAIL (Novus Biologicals)
Rabbit, Polyclonal (NB100-2056), IgG, Immunogen: Synthetic peptide
(Human) (C terminal) (Novus Biologicals)
Mouse, Monoclonal (NBP1-40925), IgG1, Immunogen: Recombinant human
CD253 (TRAIL) aa 95-281 (Novus Biologicals)
Mouse, Monoclonal (NB 120-105160) IgG1, Immunogen: Recombinant full
length protein (Human), (Novus Biologicals)
Mouse, Monoclonal (NBP1-97640), IgG2b, clone: III6F, Immunogen:
Recombinant human soluble TRAIL (Novus Biologicals)
Mouse, Monoclonal (NBP1-40925APC, NBP1-40925B, NBP1-40925F), IgG1,
Clone: 2E5, Immunogen: Recombinant human CD253 (TRAIL) aa 95-281
(Novus Biologicals)
Mouse, Monoclonal (LS-C18675-100) IgG2b (LifeSpan BioSciences)
Mouse, Monoclonal (LS-C104433-500) IgG1 (LifeSpan BioSciences)
Rabbit, polyclonal (LS-C193075-100, LS-C343632-100, LS-C48155-100, LS-
C48155-30) (LifeSpan BioSciences)
Rabbit, Polyclonal (LS-C331936-50, LS-C331936-200, LS-C331936-100, LS-
C330931-50, LS-C330931-200), IgG (LifeSpan BioSciences
Mouse, Monoclonal (LS-C18676-100), IgG2 (LifeSpan BioSciences)
Mouse, Monoclonal (LS-C 18129-500), IgG1 kappa, (LifeSpan BioSciences)
Rabbit, Polyclonal (LS-C147115-100, LS-C147115-30, LS-C137410-100, LS-
C211863-100, LS-C211863-50, LS-C211860-100, LS-C211860-50, LS-B7420-
50, LS-C40709-100, LS-C10819-50, LS-C10819-100), IgG (LifeSpan
BioSciences)
Chicken, Polyclonal (LS-C96173-100) (LifeSpan BioSciences)
Mouse, monoclonal (LS-C18671-100), IgG2a (LifeSpan BioSciences)
Rabbit, Polyclonal (LS-C153060-100, LS-C193075-100, LS-C104497-25, LS-
C104497-50, LS-B633-50) (LifeSpan BioSciences)
Rabbit, Polyclonal (LS-C51715-50, LS-C254-100, LS-C10810-100)
Immunogen: C-Terminus, (LifeSpan BioSciences)
Rabbit, Polyclonal (LS-C386509-100), IgG, aal-80, (LifeSpan BioSciences)
Rabbit, Polyclonal (LS-C296890-100), IgG, aa114-281, (LifeSpan BioSciences)
Mouse, Monoclonal (LS-C292534-200), IgG, aa114-281, (LifeSpan
BioSciences)
Mouse, Monoclonal (LS-C41050-100, LS-C106823-100, LS-C106823-25, LS-
C106723-25, LS-C106723-100, LS-C105978-100, LS-C105978-25), IgG1,
clone: RIK-2, (LifeSpan BioSciences)
Rabbit, Polyclonal (LS-C298957-100, LS-C304925-100), IgG, Immunogen:
aa114-281, (LifeSpan BioSciences)
Mouse, Monoclonal (LS-C298958-200, LS-C304927-200), IgG, Immunogen:
aa114-281, (LifeSpan BioSciences)
Rabbit, Polyclonal (LS-C40612-100), IgG, Immunogen: aal33-144 Residues
SNTLSSPNSKNE - SEQ ID NO: 112 of the N terminus of the TRAIL protein,
(LifeSpan BioSciences)
Rabbit, Polyclonal (LS-C90682-1000) Immunogen: aal7-35, (LifeSpan
BioSciences)
Rabbit, Polyclonal (LS-C49326-100) Immunogen: aa223-235, Reacts with
residues MKSARNSCWSKDA - SEQ ID NO: 110 of the C terminus of the
TRAIL protein (LifeSpan BioSciences)
Rabbit, Polyclonal (LS-C30023-50), IgG, Immunogen: aa56-105, (LifeSpan
BioSciences)
Mouse, Monoclonal (LS-C10809-100), IgG1, Immunogen: aa95-281, (LifeSpan
BioSciences)
Mouse, Monoclonal (LS-C349915-500, LS-C349914-500, LS-C349913-500),
IgG2b, Clone: SB91a, (LifeSpan BioSciences)
Mouse, Monoclonal (LS-C493-100), IgG, Immunogen: aa17-35, Clone:
55B709.3, (LifeSpan BioSciences)
Rabbit, Polyclonal (LS-B7802-50), IgG, aa31-80, (LifeSpan BioSciences)
Mouse, Monoclonal (LS-C204834-100), IgG1, Immunogen: aa95-281, Clone
2E5, (LifeSpan BioSciences)
Mouse, Monoclonal (LS-C191608-500), IgG1 Immunogen: aa95-281, Clone
366G/76/3, (LifeSpan BioSciences)
Rabbit, Polyclonal (LS-C417100-100, LS-C415440-100, LS-C415053-100),
(LifeSpan BioSciences)
Rabbit, Polyclonal (LS-C489552-100, LS-C489552-400, LS-C489552-200, LS-
C412015-100, LS-C489553-50, LS-C489553-200, LS-C489553-100, LS-
C489550-200, LS-C489550-100, LS-C489550-50), (LifeSpan BioSciences)
Mouse, Monoclonal (LS-C489549-50, LS-C489549-100, LS-C489549-200),
(LifeSpan BioSciences)
Mouse, Monoclonal (LS-C511776-100, LS-C519622-100, LS-C539797-100,
LS-C549967-100, LS-C549967-100, LS-C657939-100, LS-C529880-100), IgG1
k, (LifeSpan BioSciences)
Rabbit, Polyclonal (LS-C511772-100, LS-C511775-100, LS-C519623-100, LS-
C529882-100, LS-C539800-100, LS-C539799-100, LS-C549964-100, LS-
C549968-100, LS-C211893-50, LS-C657937-100, LS-C657940-100, LS-
C10814-100), IgG, (LifeSpan BioSciences)
Rabbit, Polyclonal (LS-C529878-100), IgG k, (LifeSpan BioSciences)
Rabbit, Polyclonal (LS-C387279-100), IgG, Immunogen: aa31-80, (LifeSpan
BioSciences)
Rabbit, Polyclonal (LS-C481142-100, LS-C481143-100, LS-C481144-100),
IgG, Immunogen: aa56-105, (LifeSpan BioSciences)
Rabbit, Polyclonal (OAPB00009) IgG (Aviva Systems Biology)
Rabbit, Polyclonal (BML-SA259-0100) (Enzo Life Sciences, Inc.)
Rabbit, Polyclonal (ALX-210-732-R100) Immunogen: Recombinant human
soluble TRAIL (aa 95-281)) (Enzo Life Sciences, Inc.)
Rabbit, Polyclonal (ADI-AAP-470-E) Immunogen: Synthetic peptide
corresponding to the sequence near the C-terminus of human TRAIL (Enzo Life
Sciences, Inc.)
Mouse, Monoclonal (ALX-804-296-C100) IgG1, Immunogen: Recombinant
human soluble TRAIL (aa 95-281) (Enzo Life Sciences, Inc.)
Mouse, Monoclonal (ALX-804-300-C100) IgG1, Clone: HS501, Immunogen:
Recombinant human soluble TRAIL, (Enzo Life Sciences, Inc.)
Mouse, Monoclonal (ALX-804-326-C100), IgG2b, Clone: III6F, Immunogen:
Recombinant human soluble TRAIL, (Enzo Life Sciences, Inc.)
Mouse, Monoclonal (ALX-804-325-C100), IgG2b, Clone: VI10E, Immunogen:
Recombinant human soluble TRAIL, (Enzo Life Sciences, Inc.)
Mouse, Monoclonal (ALX-804-907-0100) IgG1, Clone: RIK-2, Immunogen:
Human TRAIL transfected mouse cell line (Enzo Life Sciences, Inc.)
Rat, Monoclonal (MABC12) IgG2a kappa (MilliporeSigma)
Mouse, Monoclonal (MABC147), IgG1a kappa, Clone: 6D12.2, Immunogen:
GST-tagged recombinant protein corresponding to human TRAIL/CD253,
(MilliporeSigma)
Rabbit, Polyclonal (AP00024PU-N, AP06701PU-N) (OriGene Technologies)
Rabbit, Polyclonal (PP1084B1, PP1084B2, PP1084P1) (OriGene Technologies)
Mouse, Monoclonal (PM1214P), IgG1, (OriGene Technologies)
Mouse, Monoclonal (AM31199BT-N, AM31199PU-N, AM31199AF-N,
AM31199RP-N, AM31323AF-N), IgG1, Clone: B-S23, (OriGene
Technologies)
Mouse, Monoclonal (AM31345BT-N, AM31200AF-N) IgG1, Clone: B-T24,
(OriGene Technologies)
Mouse, Monoclonal (SM1847RP, SM1847LE, SM1847P),IgG1, Clone: 2E5,
(OriGene Technologies)
Rabbit, Polyclonal (SP1177P, SP1177S, TA305942) IgG, (OriGene
Technologies)
Rabbit, polyclonal (STJ96876, STJ25899) IgG (St John's Laboratory)
Mouse, monoclonal (11050-01, 11040-01) IgG2b (SouthernBiotech)
Rabbit Monoclonal (10465-R102-P) IgG (Sino Biological)
Mouse Monoclonal (10408-MM07) IgG2a (Sino Biological)
Mouse, Monoclonal (10409-MM03) IgG1, Clone: 03 (Sino Biological)
Rabbit, Polyclonal (10409-RP01, 10409-RP01, 101008-T08, 10409-RP02) IgG
(Sino Biological)
Rabbit, Polyclonal (XP-5289, XP-5289Bt, 31-018) (ProSci, Inc)
Rabbit, Polyclonal (1113), IgG, Immunogen: a peptide corresponding to 17
amino acids near the carboxy terminus of human TRAIL. (ProSci, Inc)
Mouse, Monoclonal (XP-5726-M) IgG1 kappa, Clone: 1.1_1A7-2B7 (ProSci,
Inc)
Mouse, Monoclonal (XP-5729-M) IgG kappa, Clone: M66-4B (ProSci, Inc)
Mouse, Monoclonal (36-136) IgG1, Clone: HS501 (ProSci, Inc)
Rabbit, Polyclonal (T880-63R-100) IgG (SignalChem)
Mouse, Monoclonal (66756-1-Ig) IgG1, Clone: 1B9B4 (Proteintech Group Inc)
Rabbit, Polyclonal (17235-1-AP) IgG (Proteintech Group Inc)
Mouse, Monoclonal (GWB-C86B93) IgG1 (GenWay Biotech, Inc.)
Mouse, Monoclonal (GWB-391EFA) IgG1 (GenWay Biotech, Inc.)
Rabbit, Polyclonal (226436-100 ul/ 50 ul) (United States Biological)
Synthetic peptide corresponding to amino acids 50-100 of Human TRAIL
Rabbit, Polyclonal (T8180-01J-1 mg, T8180-01F-100 ug) Immunogen: Synthetic
peptide (VLIVIFTVLLQSLCVAVTY - SEQ ID NO: 113) corresponding to
aa17-35 of human TRAIL (United States Biological)
Rabbit, Polyclonal (T8180-01L-100 ug) IgG, Immunogen: Recombinant human
CD253 (United States Biological)
Rabbit, Polyclonal (T8180-01G-100 ug, 136489-50 ul, T8180-01-100 ug) (United
States Biological)
Mouse, Monoclonal (T8180-01V-500 ug, 214619-100 ug) IgG1, Clone: 2E5
(United States Biological)
Rabbit, Polyclonal (T8180-07D-100 ug, T8180-06C-100 ug) IgG, Immunogen:
Recombinant corresponding to human TRAIL expressed in E. coli (P50591)
(United States Biological)
Rabbit, Polyclonal (T8180-07B-100 ug) IgG, Immunogen: Synthetic peptide
corresponding to the human TRAIL protein (United States Biological)
Chicken, Polyclonal (T8180-04D-50 ug) IgY, Immunogen: Synthetic peptide
corresponding to aal39-281 of Human TRAIL. (United States Biological)
Mouse, Monoclonal (T8180-04C-100 ug) IgG, Immunogen: peptide
corresponding to amino acids 17-35 of human TRAIL. (United States
Biological)
Mouse, Monoclonal (T8175-04C-100 ug) IgG2b, Immunogen: Recombinant
human soluble TRAIL (United States Biological)
Mouse, Monoclonal (T8175-02A-100 ) IgG1, Clone: 6D806, Immunogen:
Recombinant human soluble TRAIL (aa 95-281) (United States Biological)
Mouse, Monoclonal (T8175-04-100 ug) IgG1, Clone: 6D807, Immunogen:
Recombinant human soluble TRAIL (United States Biological)
Mouse, Monoclonal (045345-100 ug) IgG1, Clone: HS501 (United States
Biological)
Mouse, Monoclonal (T8175-04F-100 ug) IgG2b, Clone: 6D809, Immunogen:
Recombinant human soluble TRAIL (United States Biological)
Mouse, Monoclonal (T8180-04-500 ug) IgG1 kappa, Clone: 41170 (United States
Biological)
Rabbit, Polyclonal (T8180-05-100 ug), Immunogen: Highly purified,
recombinant human TRAIL/Apo2L (United States Biological)
Rabbit, Polyclonal (T8175-08-100 ul, T8180-05-50 ug) IgG, Immunogen:
Recombinant human soluble TRAIL (aa 95-281) (United States Biological)
Rabbit, Polyclonal (T8180-01-50 ug) IgG, Immunogen: Human TRAIL protein
(United States Biological)
Rabbit, Polyclonal (T8175-05-100 ug) IgG, Immunogen: An 18 residue synthetic
peptide CTNEHLIDMDHEASFFGA - SEQ ID NO: 114 from the carboxy
terminal region of human TRAIL (residues 261-277) (United States Biological)
Rabbit, Polyclonal (T8180-06-50 ug, T8180-06-25 ug) IgG, Immunogen:
Recombinant human TRAIL (Highly purified, > 98%) (United States
Biological)
Rabbit, Polyclonal (T8180-02-100 ug, T8180-02-50 ug) IgG, Immunogen:
Synthetic peptide, aa[MKSARNSCWSKDA - SEQ ID NO: 110] of the C
terminus of the TRAIL protein (United States Biological)
Rabbit, Polyclonal (T8175-03-100 ug) IgG, Immunogen: Peptide from C-
terminus of human TRAIL (United States Biological)
Rabbit, Polyclonal (T8180-03-100 ug, T8180-03-50 ug) Immunogen: Synthetic
peptide, aa[SNTLSSPNSKNE - SEQ ID NO: 112] of the N terminus of the
TRAIL protein (United States Biological)
Rabbit, Polyclonal (226390-50 ul, 226390-100 ul) Immunogen: Recombinant full
length Human TRAIL (United States Biological)
Rabbit, Polyclonal (T8175-100 ug) Immunogen: Peptide corresponding to amino
acid 261 to 277 of human TRAIL (United States Biological)
Mouse, Polyclonal (134545-50 ug) IgG, Immunogen: Full length human
TNFSF10, aa1-300 (United States Biological)
Rabbit, Polyclonal (134546-100 ug) IgG, Immunogen: Full length human
TNFSF10, aal-300 (United States Biological)
Rabbit, Polyclonal (337741-50 ug, 337742-50 ug, 337741-100 ug, 337742-100 ug)
Immunogen: Synthetic peptide corresponding to the Internal region of human
TRAIL (United States Biological)
Rabbit, Polyclonal (338581-150 ul, 338581-50 ul, 338517-50 ug, 347111-200 ul,
347111-100 ul, 346321-200 ul, 361342-100 ul 338517-100 ug, 361342-50 ul,
338518-50 ug, 338518-100 ug, 338519-50 ug, 338519-100 ug, 338520-100 ug,
171357-50 ug, 213851-50 ul, 213851-100 ul) (United States Biological)
Rabbit, Polyclonal (346321-200 ul) Immunogen: Synthesized peptide derived
from the Internal region of human TRAIL, aa1-80 (United States Biological)
Mouse, Monoclonal (142701-200 ug) Immunogen: TRAIL (Val114-Gly281)
(United States Biological)
Mouse, Monoclonal (214624-25 ug) IgG1, Clone: DJR2-4 (United States
Biological)
Mouse, Monoclonal (217026-100 ug) IgG1, Clone: 14L929 (United States
Biological)
Mouse, Monoclonal (228258-500 ug) IgG1, Clone: SB91c (United States
Biological)
Mouse, Monoclonal (228257-500 ug, 228259-500 ug) IgG1, Clone: SB91a
(United States Biological)
Rabbit, Polyclonal (142700-100 ug, 142700-50 ug) IgG, Immunogen: TRAIL
(Val114-Gly281) (United States Biological)
Mouse, monoclonal (563642, 564243, 550431, 550516, 561784, 550912,
743720, 743721, 743722, 743723) IgG1, Clone: RIK-2 (BD Biosciences)
Mouse, monoclonal (550517) IgG1, Clone: RIK-1 (BD Biosciences)
Mouse, Monoclonal (556468) IgG2b kappa, Clone: B35-1 (BD Biosciences)
Rabbit, polyclonal (500-P135-50 UG, 500-P135-100 UG, 500-P135BT-50 UG,
500-P135BT-25 UG) (PeproTech)
Mouse, Monoclonal (500-M49-500 UG) IgG1 kappa (PeproTech)
Rabbit, Polyclonal (A00466-1, A00466-2) (BosterBio)
Rabbit, Polyclonal (BS2007) Immunogen: amino acids 50-100 of Human
TRAIL (Bioworld Technology)
Rabbit, Polyclonal (BS6907, BS60933) Immunogen: Recombinant full length
Human TRAIL (Bioworld Technology)
Rabbit, Polyclonal (ABIN1003406, ABIN673494) IgG (antibodies-online)
Rabbit, Polyclonal (AB INI 172417, ABIN1172416, ABIN501030,
ABIN462651, ABIN1046206, ABIN1996723, ABIN2153710, ABIN2153461,
ABIN1740255, ABIN1172416, ABIN1003406, ABIN2379261, ABIN2379243,
ABIN2479502, ABIN2770031, ABIN222899, ABIN643994, ABIN481110,
ABIN121515, ABIN1046207, ABIN1094086, ABIN640733, ABIN214907,
ABIN1955942, ABIN2153711, ABIN205219, ABIN1820762, ABIN2879096)
IgG (antibodies-online)
Mouse, Monoclonal (ABIN1106147, ABIN1106154, ABIN1383744,
ABIN1721267, ABIN1722562, ABIN1722442, ABIN1106148) IgG1, Clone: B-
S23 (antibodies-online)
Mouse Monoclonal (ABIN1106152, ABIN1106149, ABIN1383747) IgG1,
Clone: B-T24 (antibodies-online)
Rabbit, polyclonal (ABIN1104738) IgG, Immunogen: E. coli derived
recombinant Human TRAIL/Apo2L. (antibodies-online)
Rabbit, Polyclonal (ABIN1585442, ABIN926879, ABIN2705773,
ABIN2767835, ABIN2858501, ABIN201278, ABIN636583, ABIN272200,
ABIN802647, AB INI 16275, ABIN116273, ABIN1583435, ABIN1849410,
ABIN151524, ABIN2892121, ABIN241624) (antibodies-online)
Mouse, Monoclonal (ABIN2379229) Immunogen: Recombinant human soluble
TRAIL (aa 95-281) (antibodies-online)
Rabbit, Polyclonal (ABIN2377666) IgG, Immunogen: Synthetic peptide
corresponding to the internal region of human TNFSF10/TRAIL (antibodies-
online)
Mouse, Monoclonal (ABIN2224540, ABIN198205) IgG1, Immunogen:
Recombinant protein corresponding to aa95-281 from human CD253
(antibodies-online)
Rabbit, Polyclonal (ABIN1740453) IgG, Immunogen: residues
[MKSARNSCWSKDA] of the C terminus of the TRAIL protein (antibodies-
online)
Mouse, Monoclonal (ABIN1172418) ( antibodies-online)
Rabbit, Polyclonal (ABIN1078632) IgG, Immunogen: TRAIL (Val114-Gly281)
(antibodies-online)
Mouse, Monoclonal (ABIN1860863) IgG, Immunogen: TRAIL (AA 114-281)
(antibodies-online)
Rabbit, Polyclonal (ABIN2904425) Immunogen: TRAIL (Val114-Gly281)
(antibodies-online)
Mouse, Monoclonal (ABIN2379245, ABIN2479500, ABIN2749022,
ABIN1942975) IgG1, Clone: 2.00E+05 (antibodies-online)
Mouse, Monoclonal (ABIN2379231), IgG, Clone: 6D807, Immunogen:
Recombinant human soluble TRAIL (antibodies-online)
Chicken, Polyclonal (ABIN2379258) IgY (antibodies-online)
Mouse, Monoclonal (ABIN2379250) IgG1 kappa, Clone: 41170 (antibodies-
online)
Rabbit, Polyclonal (ABIN2379235) IgG, Immunogen: Recombinant human
soluble TRAIL (aa 95-281) (antibodies-online)
Mouse, Monoclonal (ABIN2379257) IgG, Clone: 4H289, Immunogen: peptide
corresponding to amino acids 17-35 of human TRAIL (antibodies-online)
Mouse, Monoclonal (ABIN2668580, ABIN207859) IgG1, Clone: 55B709-3
(antibodies-online)
Mouse, Monoclonal (ABIN2682692) IgG1, Clone: 3 (antibodies-online)
Mouse, Monoclonal (ABIN199858, ABIN199859) IgG2b (antibodies-online)
Mouse, Monoclonal (ABIN473970, ABIN262794) IgG1 kappa, Clone: RIK-2
(antibodies-online)
Mouse, Polyclonal (ABIN563764) Immunogen: aa 1-281 (antibodies-online)
Mouse, Polyclonal (ABIN522187) Immunogen: aa 1-300 (antibodies-online)
Rabbit, Polyclonal (ABINI 104737), Immunogen: aa 261-277 (antibodies-
online)
Mouse, Monoclonal (ABIN115786) IgG1 (antibodies-online)
Chicken, Polyclonal (ABIN379904) (antibodies-online)
Goat, Polyclonal (ABIN238961) (antibodies-online)
Mouse, Monoclonal (ABIN187524) IgG2b, Clone: IIIF6 (antibodies-online)
Mouse, Monoclonal (ABIN933806), IgG1 kappa, Clone: M912292 (antibodies-
online)
Rabbit, Polyclonal (ABIN216393) IgG, Immunogen: aa56-105 (antibodies-
online)
Mouse, Monoclonal (ABIN1169389) IgG1, Clone: HS501 (antibodies-online)
Mouse, Monoclonal (ABIN1003307) IgG1 kappa, (antibodies-online)
Mouse, Monoclonal (ABINI823246) IgG1, Clone: 366G-76-3 (antibodies-
online)
Mouse, Monoclonal (ABIN1983464) IgG1, Clone: MM0580-6D44,
Immunogen: aa 95-281 (antibodies-online)
Mouse, Monoclonal (ABIN265772), IgG1, Clone: 75411-11 (antibodies-online)
Mouse, Monoclonal (ABIN470647) IgG2 (antibodies-online)
Rabbit, Polyclonal (168-10922) IgG (Raybiotech, Inc.)
Rabbit, Polyclonal (168-10034) (Raybiotech, Inc.)
Rabbit, (119-11761, 119-16686) (Raybiotech, Inc.)
Rabbit, Polyclonal (C01766B, C01766F, C01766H, C01766Cy3, C01766Cy5)
IgG (Signalway Antibody LLC)
Rabbit, Polyclonal (41881) (Signalway Antibody LLC)
Rabbit, Polyclonal (E-AB-30256) IgG, Immunogen: Synthesized peptide
derived from the Internal region of human TRAIL (Elabscience Biotechnology
Inc.)
Rabbit, Polyclonal (E-AB-33140) IgG, Immunogen: Synthesized peptide
derived from the Internal region of human TRAIL (Elabscience Biotechnology
Inc.)
Mouse, Monoclonal (10-1012) IgG1 kappa, Immunogen: recombinant human
TRAIL protein (amino acids 10-230) (Abeomics)
Rabbit, Polyclonal (2430) Immunogen: aa 261-277 of human TRAIL (QED
Bioscience Inc.)
Mouse, Monoclonal (1P-316-C100; 1P-316-C025; 12-316-C100, 11-316-C100,
11-316-C025) IgG1, Clone: 2E5, Immunogen: Recombinant soluble fragment
(aa 95-281) of human TRAIL (EXBIO Praha, a.s.)
Mouse, Monoclonal (130-097-314, 130-097-301, 130-097-304) IgG1, Clone:
RIK-2.1, Miltenyi Biotec
Mouse, Monoclonal (MAB8218) IgG2b, Clone: SB91a (Abnova Corporation)
Mouse, Monoclonal (MAB8219) IgG2b, Clone: SB91c (Abnova Corporation)
Rabbit, Polyclonal (PAB24882, PAB0842, PAB12822, H00008743-D01P)
(Abnova Corporation)
Mouse, Polyclonal (H00008743-A01, H00008743-B01P) (Abnova Corporation)
Mouse (MC670320) IgG1 (Antigenix America Inc.)
Rabbit, Polyclonal (RHF760) (Antigenix America Inc.)
Rabbit, Polyclonal (RHF760B) (Antigenix America Inc.)
Mouse, Monoclonal (CAU29201) Immunogen: Val114-Gly2810 (Biomatik)
Rabbit, Polyclonal (CAU28486) Immunogen: Val114-Gly281 (Biomatik)
Mouse, Monoclonal (CDM079; CDM077; CDM078, CDM076) IgG1, Clone B-
S23 (Cell Sciences)
Mouse, Monoclonal (CDM080) IgG1, Clone: B-T24 (Cell Sciences)
Mouse, Monoclonal (CDM415, CDM439) (Cell Sciences)
Rabbit, Polyclonal (PX065A, PA 1322) (Cell Sciences)
Rabbit, Polyclonal (abx104658) (Abbexa Ltd)
Mouse, Monoclonal (abx104659) IgG2b kappa, Immunogen: Val114-Gly281
(Abbexa Ltd)
Mouse, Monoclonal (CLX106NA) IgG1, Clone 2E5 (CEDARLANE)
Mouse, Monoclonal (MCA2144EL; MCA2144ELGA) IgG1, Clone 2E5,
Immunogen: Recombinant human TRAIL aa 95-281 (Bio-Rad )
Rabbit, Polyclonal (AHP1215) IgG, Immunogen: Recombinant human CD253
(Bio-Rad)
Rabbit, Polyclonal (AHP440) IgG, Immunogen: Peptide from C-terminus of
human TRAIL (Bio-Rad)
Rabbit, Polyclonal (HPA054938) IgG (Atlas Antibodies)
Mouse, Monoclonal (AFC-4091-1; AFC-4626-2; AFC-4091-2; AFC-4247-2;
AFC-5159-2; AFC-5159-1) Clone 2E5. Immunogen: Recombinant soluble
fragment (aa 95-281) of human TRAIL (Nordic BioSite)
Rabbit, Polyclonal (AMS.ENT4721; AMS.ENK5511; PCA-2135a-9;
AMS.ENT4721; AMS.ENK5511) IgG, Immunogen: Synthesized peptide
derived from the Internal region of human TRAIL (AMSBIO LLC)
Mouse, Monoclonal (879.770.001, 853.083.020), IgG1, Clone B-S23 (Sapphire
North America)
Mouse, Monoclonal (879.770.002, 853.090.000), IgG1, clone B-T24 (Sapphire
North America)
Monoclonal, (LAA139Hu72, LAA139Hu82), (Cloud-Clone)
Polyclonal (LAA139Hu71, LAA139Hu82) (Cloud-Clone)
Mouse, Monoclonal (MAA139Hu22), IgG2b Kappa (Cloud-Clone)
Rabbit, Polyclonal (PAA139Hu01) (Cloud-Clone)
Mouse, Monoclonal (sc-8440), Clone D-3, (Santa Cruz Biotechnology, Inc.)
Mouse, Monoclonal (sc-56246) Clone: RIK-2 (Santa Cruz Biotechnology, Inc.)
Mouse, Polyclonal (DPAB-DC3637) (Creative Diagnostics)
Mouse, Monoclonal (DCABH-7664) IgG1, Clone 3F6 (Creative Diagnostics)
Mouse, Monoclonal (DCABH-5643), IgG1, Clone SJL-3 (Creative Diagnostics)

The TRAIL epitopes of the antibodies of the present invention are in any of the sequences as set forth in SEQ ID NOs: 1-95. It will be appreciated that the antibody may bind to one, two, three or four of the amino acids of SEQ ID NOs: 1-95, wherein the sequences are at positions as shown in FIG. 5 (blue, green and red boxes).

RGRS (SEQ ID NO: 10), GRSN (SEQ ID NO: 11), RSNT (SEQ ID NO: 12), SNTL (SEQ ID NO: 13), NTLS (SEQ ID NO: 14), TLSS (SEQ ID NO: 16), LSSP (SEQ ID NO: 17), SSPN (SEQ ID NO: 18), SPNS (SEQ ID NO: 19), PNSK (SEQ ID NO: 20), NSKN (SEQ ID NO: 21), SKNE (SEQ ID NO: 22), KNEK (SEQ ID NO: 23), NEKA (SEQ ID NO: 24), EKAL (SEQ ID NO: 25), KALG (SEQ ID NO: 26), ALGR (SEQ ID NO: 27), LGRK (SEQ ID NO: 28), GRKI (SEQ ID NO: 29), RKIN (SEQ ID NO: 30), FRFQ (SEQ ID NO: 31), RGRSN (SEQ ID NO: 32), GRSNT (SEQ ID NO: 33), RSNTL (SEQ ID NO: 34), SNTLS (SEQ ID NO: 35), NTLSS (SEQ ID NO: 36), TLSSP (SEQ ID NO: 37), LSSPN (SEQ ID NO: 38), SSPNS (SEQ ID NO: 39), SPNSK (SEQ ID NO: 40), PNSKN (SEQ ID NO: 41), NSKNE (SEQ ID NO: 42), SKNEK (SEQ ID NO: 43), KNEKA (SEQ ID NO: 44), NEKAL (SEQ ID NO: 45), EKALG (SEQ ID NO: 46), KALGR (SEQ ID NO: 47), ALGRK (SEQ ID NO: 48), LGRKI (SEQ ID NO: 49), GRKIN (SEQ ID NO: 50), RGRSNT (SEQ ID NO: 51), RSNTLS (SEQ ID NO: 53), SNTLSS (SEQ ID NO: 54), NTLSSP (SEQ ID NO: 55), TLSSPN (SEQ ID NO: 56), LSSPNS (SEQ ID NO: 57), SSPNSK (SEQ ID NO: 58), SPNSKN (SEQ ID NO: 59), PNSKNE (SEQ ID NO: 60), NSKNEK (SEQ ID NO: 61), SKNEKA(SEQ ID NO: 62), KNEKAL (SEQ ID NO: 63), NEKALG (SEQ ID NO: 64), EKALGR (SEQ ID NO: 65), KALGRK (SEQ ID NO: 66), ALGRKI (SEQ ID NO: 67), LGRKIN (SEQ ID NO: 68), EEIK (SEQ ID NO: 69), EIKE (SEQ ID NO: 70), IKEN (SEQ ID NO: 71), KENT (SEQ ID NO: 72), ENTK (SEQ ID NO: 73), NTKN (SEQ ID NO: 74), TKND (SEQ ID NO: 75), KNDK (SEQ ID NO: 76), NDKQ (SEQ ID NO: 77), DKQM (SEQ ID NO: 78), EEIKE (SEQ ID NO: 79), EIKEN (SEQ ID NO: 80), IKENT (SEQ ID NO: 81), KENTK (SEQ ID NO: 82), ENTKN (SEQ ID NO: 83), NTKND (SEQ ID NO: 84), NTKNDK (SEQ ID NO: 85), TKNDKQ (SEQ ID NO: 86), KNDKQM (SEQ ID NO: 87), EEIKEN (SEQ ID NO: 88), EIKENT (SEQ ID NO: 89), IKENTK (SEQ ID NO: 90), KENTKN (SEQ ID NO: 91), ENTKND (SEQ ID NO: 92), NTKNDK (SEQ ID NO: 93), TKNDKQ (SEQ ID NO: 94), KNDKQM (SEQ ID NO: 95).

It will be appreciated that the epitopes of the antibodies of the present invention may be longer than those set forth in SEQ ID NOs: 1-95. Thus, for example, the full-length epitope may be in one of the sequences as set forth in SEQ ID NOs: 97, 98 and 101-109.

As mentioned, in one embodiment, the antibody is a monoclonal antibody (e.g. a recombinant monoclonal antibody). The monoclonal antibody may bind to any of the epitopes described herein including for example those comprised in SEQ ID NO: 4 or 74. In a particular embodiment, the antibody binds to the TRAIL protein at the sequence 4, 74, 97 or 98

In another embodiment, the antibody is a polyclonal antibody which recognizes at least one, at least two, at least three, at least four, at least five, at least six, at least seven, at least eight, at least nine or more of the epitopes described herein.

Preferably, at least one, at least two, at least three, at least four, at least five, at least six, at least seven, at least eight of the epitopes detected by the polyclonal antibody is set forth in Table 4 of the examples section herein below. The extended sequences of the epitopes are set forth in Table 5 of the Examples section below, which are also contemplated by the present invention.

In a particular embodiment, the polyclonal antibody does not recognize all of the epitopes listed in Table 4.

The antibodies of the present invention are useful for detecting TRAIL in biological samples.

Thus, according to an aspect of the present invention, there is provided a method of detecting TRAIL in a biological sample comprising:

(a) contacting the sample with the antibody described herein; and

(b) detecting said at least one antibody.

The term “detecting”, as used herein, refers to the act of detecting, perceiving, uncovering, exposing, visualizing or identifying TRAIL. Preferably, the detecting is effected such that the amount of TRAIL present in the sample can be quantified. The precise method of detecting is dependent on the detectable moiety (also referred to herein as identifiable moiety) to which the antibody is attached as further described herein below.

In one embodiment, the detectable signal is observed in about ten minutes or less following the contacting step.

In another embodiment, the detectable signal is observed in about 5 minutes or less following the contacting step.

In still another embodiment, the detectable signal is observed no longer than 3 minutes following the contacting step.

A “sample” in the context of the present invention is a biological sample isolated from a subject and can include, by way of example and not limitation, whole blood, serum, plasma, saliva, mucus such as nasal mucus which has been collected using a nasal swab, breath, urine, CSF, sputum, sweat, stool, hair, seminal fluid, biopsy, rhinorrhea, tissue biopsy, cytological sample, platelets, reticulocytes, leukocytes, epithelial cells, or whole blood cells.

In a particular embodiment, the sample is a blood sample—e.g. serum, plasma, whole blood. The sample may be a venous sample, capillary blood sample, peripheral blood mononuclear cell sample or a peripheral blood sample. Preferably, the sample comprises white blood cells including for example granulocytes, lymphocytes and/or monocytes. In one embodiment, the sample is depleted of red blood cells.

In one embodiment, the antibody of this aspect of the present invention is able to detect TRAIL at very low levels in a blood sample, for example when the TRAIL is present at 70 pg/ml, at 60 pg/ml, at 50 pg/ml, at 40 pg/ml, at 30 pg/ml and even at 20 pg/ml as determined by an immunoassay (such as those described herein).

The sample may be fresh or frozen. Preferably, the level of TRAIL is measured within about 6 hours, 12 hours or 24 hours after the sample is obtained. Alternatively, the concentration of the polypeptides is measured in a sample that was stored at 12 degrees C. or lower.

The contacting may be effected in vitro (i.e. in a cell line, primary cells), ex vivo or in vivo.

As mentioned, the method of the present invention is effected under conditions sufficient to form an immunocomplex (e.g. a complex between at least one antibody of the present invention and TRAIL).

Determining a presence or level of the immunocomplex of the present invention is dependent on the detectable moiety to which the antibody is attached.

Examples of detectable moieties that can be used in the present invention include but are not limited to radioactive isotopes, phosphorescent chemicals, chemiluminescent chemicals, fluorescent chemicals, enzymes, fluorescent polypeptides and epitope tags. The detectable moiety can be a member of a binding pair, which is identifiable via its interaction with an additional member of the binding pair, and a label which is directly visualized. In one example, the member of the binding pair is an antigen which is identified by a corresponding labeled antibody. In one example, the label is a fluorescent protein or an enzyme producing a colorimetric reaction.

Further examples of detectable moieties include those detectable by Positron Emission Tomagraphy (PET) and Magnetic Resonance Imaging (MRI), all of which are well known to those of skill in the art.

When the detectable moiety is a polypeptide, the immunolabel (i.e. the antibody conjugated to the detectable moiety) may be produced by recombinant means or may be chemically synthesized by, for example, the stepwise addition of one or more amino acid residues in defined order using solid phase peptide synthetic techniques. Examples of polypeptide detectable moieties that can be linked to the antibodies of the present invention using recombinant DNA technology (in which the polynucleotide encoding the TCRL is translationally fused to the detectable moiety) include fluorescent polypeptides, phosphorescent polypeptides, enzymes and epitope tags.

Alternatively, chemical attachment of a detectable moiety to the antibodies of the present invention can be effected using any suitable chemical linkage, direct or indirect, as via a peptide bond (when the detectable moiety is a polypeptide), or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched or cyclic side chains, internal carbon or nitrogen atoms, and the like. Such modified peptides can be easily identified and prepared by one of ordinary skill in the art, using well known methods of peptide synthesis and/or covalent linkage of peptides. Description of fluorescent labeling of antibodies is provided in details in U.S. Pat. Nos. 3,940,475, 4,289,747, and 4,376,110.

According to a specific embodiment, the detection is effected by an immunoassay.

Immunoassays carried out in accordance with some embodiments of the present invention may be homogeneous assays or heterogeneous assays. In a homogeneous assay the immunological reaction usually involves the TRAIL antibody, a labeled TRAIL, and the sample of interest. The signal arising from the label is modified, directly or indirectly, upon the binding of the antibody to the labeled TRAIL. Both the immunological reaction and detection of the extent thereof can be carried out in a homogeneous solution. Immunochemical labels, which may be employed, include free radicals, radioisotopes, fluorescent dyes, enzymes, bacteriophages, or coenzymes. In a heterogeneous assay approach, the reagents are usually the sample, the primary antibody, and means for producing a detectable signal. Samples as described above may be used. The antibody (i.e. TRAIL antibody) can be immobilized on a support, such as a bead (such as protein A and protein G agarose beads), plate or slide, and contacted with the specimen suspected of containing the antigen (i.e. TRAIL) in a liquid phase. The support is then separated from the liquid phase and either the support phase or the liquid phase is examined for a detectable signal employing means for producing such signal. The signal is related to the presence of the analyte (i.e. TRAIL) in the sample. Means for producing a detectable signal include the use of radioactive labels, fluorescent labels, or enzyme labels, as described herein above.

In one embodiment, a second TRAIL antibody that binds a site other than the site to which the first antibody binds can be conjugated to a detectable group and added to the liquid phase reaction solution before the separation step. The presence of the detectable group on the solid support indicates the presence of the antigen (TRAIL) in the test sample.

In a particular embodiment, the first antibody is a monoclonal antibody (for example one that recognizes the epitope in the sequence as set forth in SEQ ID NOs: 4, 74, 97 or 98) and the second antibody is a polyclonal antibody that binds to an epitope other than the epitope to which the monoclonal antibody binds. Alternatively, the first antibody is a monoclonal antibody (for example one that recognizes the epitope in the sequence as set forth in SEQ ID NOs: 4 or 97) and the second antibody is a polyclonal antibody that binds to an epitope that overlaps the epitope of the monoclonal antibody (for example one that recognizes the epitopes in the sequences set forth in SEQ ID NO: 1-9 or 101-109). Still alternatively, the first antibody is a first monoclonal antibody (for example one that recognizes the epitope in the sequence as set forth in SEQ ID NO: 4 or 97) and the second antibody is a second monoclonal antibody that binds to an epitope other than the epitope to which the first monoclonal antibody binds (for example one that recognizes the epitope in the sequence as set forth in SEQ ID NO: 74 or 98). Still alternatively, the first antibody is a first monoclonal antibody (for example one that recognizes the epitope in the sequence as set forth in SEQ ID NO: 74 or 98) and the second antibody is a second monoclonal antibody that binds to an epitope other than the epitope to which the first monoclonal antibody binds (for example one that recognizes the epitope in the sequence as set forth in SEQ ID NO: 4 or 97). Still alternatively, the first antibody is a polyclonal antibody (for example one that recognizes the epitopes in the sequences set forth in SEQ ID NO: 1-9 or 101-109) and the second antibody is a monoclonal antibody (for example one that recognizes the epitope in the sequence as set forth in SEQ ID NO: 4, 97, 74 or 98

In the above scenarios, at least one of the antibodies used (first monoclonal antibody, second monoclonal antibody or second polyclonal antibody) is the TRAIL antibody of the present invention.

It will be appreciated that when two non-identical TRAIL antibodies are used for the detection of TRAIL (as described herein above) they may be comprised in a kit.

Each of the antibodies in the kit is typically packaged in a separate container. The containers of the kit will generally include at least one vial, test tube, flask, bottle, syringe or other containers, into which the antibody may be placed, and preferably, suitably aliquoted. The antibodies of the kit are typically provided in one or more liquid solutions, the liquid solution can be an aqueous solution. Other components of the kit may be provided as dried powder(s). When reagents and/or components are provided as a dry powder, the powder can be reconstituted by the addition of a suitable solvent.

Examples of buffers and/or washes that may be added to the kit (in order to reduce non-specific binding and increase signal to noise ratio) include high salt buffers (to exclude any non-specific binding due to ionic bonds), for example TRIS-NaCL, PBS, RIPA, HEPES-NaCL etc., detergents (to exclude any non-specific binding due to Van-Der-Vales bonds) as Dodecyl sodium sulfate (SDS), Triton-X-100, Triton-X-14, Tween-20, Tween-80, Brij, Digitonin, Cholic acid, Sodium taurodeoxycholate, CHAPSO and the same, anti-foam formulation and a preservative (a preservative can be Proclin 300, sodium azide, Thimerosal, 2-Methylisothiazol-3(2H)-one, 2-BROMO-2-NITROPROPANE-1,3-DIL.

The kit may include instructions for carrying out the immunoassay, as well as for how to prepare the kit components, and how to use any other reagent not included in the kit. Instructions may include variations on the immunoassay that can be implemented.

Examples of suitable immunoassays for detecting TRAIL using the antibodies of the present invention are immunoblotting, immunofluorescence methods, flow cytometry, radioimmunoassay, immunoprecipitation, chemiluminescence methods, electrochemiluminescence (ECL) or enzyme-linked immunoassays (ELISA).

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al., titled “Methods for Modulating Ligand-Receptor Interactions and their Application,” U.S. Pat. No. 4,659,678 to Forrest et al., titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.” The determinant can also be detected with antibodies using flow cytometry. Those skilled in the art will be familiar with flow cytometric techniques which may be useful in carrying out the methods disclosed herein (Shapiro 2005). These include, without limitation, Cytokine Bead Array (Becton Dickinson) and Luminex technology.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding.

According to a particular embodiment, the immunoassay format for detecting TRAIL is a Lateral Flow Immunoassays (LFIA). This is a technology which allows rapid measurement of analytes at the point of care (POC) and its underlying principles are described below. According to one embodiment, LFIA is used in the context of a hand-held device.

The technology is based on a series of capillary beds, such as pieces of porous paper or sintered polymer. Each of these elements has the capacity to transport fluid (e.g., urine) spontaneously.

The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex.

After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones the fluid enters the final porous material, the wick, that simply acts as a waste container. Lateral Flow Tests can operate as either competitive or sandwich assays.

Different formats may be adopted in LFIA. Strips used for LFIA contain four main components. A brief description of each is given before describing format types.

Sample application pad: It is made of cellulose and/or glass fiber and sample is applied on this pad to start assay. Its function is to transport the sample to other components of lateral flow test strip (LFTS). Sample pad should be capable of transportation of the sample in a smooth, continuous and homogenous manner. Sample application pads are sometimes designed to pretreat the sample before its transportation. This pretreatment may include separation of sample components, removal of interferences, adjustment of pH, etc.

Conjugate pad: It is the place where labeled biorecognition molecules (antibodies) are dispensed. Material of conjugate pad should immediately release labeled conjugate upon contact with moving liquid sample. Labeled conjugate should stay stable over entire life span of lateral flow strip. Any variations in dispensing, drying or release of conjugate can change results of assay significantly. Poor preparation of labeled conjugate can adversely affect sensitivity of assay. Glass fiber, cellulose, polyesters and some other materials are used to make conjugate pad for LFIA. Nature of conjugate pad material has an effect on release of labeled conjugate and sensitivity of assay.

Nitrocellulose membrane: It is highly critical in determining sensitivity of LFIA. Nitrocellulose membranes are available in different grades. Test and control lines are drawn over this piece of membrane. So an ideal membrane should provide support and good binding to capture probes (antibodies, aptamers etc.). Nonspecific adsorption over test and control lines may affect results of assay significantly, thus a good membrane will be characterized by lesser non-specific adsorption in the regions of test and control lines. Wicking rate of nitrocellulose membrane can influence assay sensitivity. These membranes are easy to use, inexpensive, and offer high affinity for proteins and other biomolecules. Proper dispensing of bioreagents, drying and blocking play a role in improving sensitivity of assay.

Adsorbent pad: It works as sink at the end of the strip. It also helps in maintaining flow rate of the liquid over the membrane and stops back flow of the sample. Adsorbent capacity to hold liquid can play an important role in results of assay.

All these components are fixed or mounted over a backing card. Materials for backing card are highly flexible because they have nothing to do with LFIA except providing a platform for proper assembling of all the components. Thus backing card serves as a support and it makes easy to handle the strip.

Major steps in LFIA are (i) preparation of antibody against target analyte (i.e. TRAIL) (ii) preparation of label (iii) labeling of biorecognition molecules (iv) assembling of all components onto a backing card after dispensing of reagents at their proper pads (v) application of sample and obtaining results.

Sandwich format: In a typical format, label (Enzymes or nanoparticles or fluorescence dyes) coated antibody is immobilized at conjugate pad. This is a temporary adsorption which can be flushed away by flow of any buffer solution. A primary antibody against TRAIL is immobilized over test line. A secondary antibody or probe against labeled conjugate antibody is immobilized at control zone.

Sample containing the analyte is applied to the sample application pad and it subsequently migrates to the other parts of strip. At conjugate pad, target analyte is captured by the immobilized labeled antibody or aptamer conjugate and results in the formation of labeled antibody conjugate/analyte complex. This complex now reaches at nitrocellulose membrane and moves under capillary action. At test line, label antibody conjugate/analyte complex is captured by another antibody which is primary to the analyte. Analyte becomes sandwiched between labeled and primary antibodies forming labeled antibody conjugate/analyte/primary antibody complex. Excess labeled antibody conjugate will be captured at control zone by secondary antibody. Buffer or excess solution goes to absorption pad. Intensity of color at test line corresponds to the amount of target analyte and is measured with an optical strip reader or visually inspected. Appearance of color at control line ensures that a strip is functioning properly.

Competitive format: Such a format suits best for low molecular weight compounds which cannot bind two antibodies simultaneously. Absence of color at test line is an indication for the presence of analyte while appearance of color both at test and control lines indicates a negative result. Competitive format has two layouts. In the first layout, solution containing target analyte is applied onto the sample application pad and prefixed labeled biomolecule (antibody/aptamer) conjugate gets hydrated and starts flowing with moving liquid. Test line contains pre-immobilized antigen (same analyte to be detected) which binds specifically to label conjugate. Control line contains pre-immobilized secondary antibody which has the ability to bind with labeled antibody conjugate. When liquid sample reaches at the test line, pre-immobilized antigen will bind to the labeled conjugate in case target analyte in sample solution is absent or present in such a low quantity that some sites of labeled antibody conjugate were vacant. Antigen in the sample solution and the one which is immobilized at test line of strip compete to bind with labeled conjugate. In another layout, labeled analyte conjugate is dispensed at conjugate pad while a primary antibody to analyte is dispensed at test line. After application of analyte solution a competition takes place between analyte and labeled analyte to bind with primary antibody at test line.

Multiplex detection format: Multiplex detection format is used for detection of more than one target species and assay is performed over the strip containing test lines equal to number of target species to be analyzed. It is highly desirable to analyze multiple analytes simultaneously under same set of conditions. Multiplex detection format is very useful in clinical diagnosis where multiple analytes which are inter-dependent in deciding about the stage of a disease are to be detected. Lateral flow strips for this purpose can be built in various ways i.e. by increasing length and test lines on conventional strip, making other structures like stars or T-shapes. Shape of strip for LFIA will be dictated by number of target analytes. Miniaturized versions of LFIA based on microarrays for multiplex detection of DNA sequences have been reported to have several advantages such as less consumption of test reagents, requirement of lesser sample volume and better sensitivity.

The high specificity of the disclosed antibodies for TRAIL in biological samples renders them particularly suitable for diagnostic applications.

Thus, according to another aspect of the present invention there is provided a method of diagnosing an infectious disease in a subject in need thereof comprising determining the amount of TRAIL in a sample (e.g. blood sample) of the subject, wherein the determining is effected using at least one antibody which comprises an antigen recognition domain that binds specifically to at least one epitope of TNF-related apoptosis-inducing ligand (TRAIL), wherein said at least one epitope is in an amino acid sequence selected from the group consisting of SEQ ID NOs: 1-95, 97, 98 and 101-109.

According to a particular embodiment, the antibody used for diagnosis is not the antibody described in Table 2.

A “subject” in the context of the present invention may be a mammal (e.g. human, dog, cat, horse, cow, sheep, pig or goat). According to another embodiment, the subject is a bird (e.g. chicken, turkey, duck or goose). According to a particular embodiment, the subject is a human. The subject may be male or female. The subject may be an adult (e.g. older than 18, 21, or 22 years or a child (e.g. younger than 18, 21 or 22 years). In another embodiment, the subject is an adolescent (between 12 and 21 years), an infant (29 days to less than 2 years of age) or a neonate (birth through the first 28 days of life).

In one embodiment, the subject of this aspect of the present invention is infectious, yet does not necessarily show symptoms of the disease—i.e. asymptomatic.

In another embodiment, the subjects of this aspect of the present invention present with symptoms of a disease.

In one embodiment, the symptoms which the subject may present with are symptoms of an infectious disease. Exemplary symptoms include but are not limited to fever, nausea, headache, sore throat, runny nose, rash and/or muscle soreness.

According to a particular embodiment, the subject does not show signs of having had a heart attack (e.g. has a normal level of creatine kinase, troponin or serum myoglobin, and/or has a normal ECG or EKG).

According to yet another embodiment, the subject does not have cancer.

In one embodiment, the level of TRAIL is used to distinguish between an infective or non-infective state.

In another embodiment, the level of TRAIL is used to distinguish between a bacterial and viral infection. In another embodiment, the level of TRAIL is used to distinguish between a bacterial and bacterial/viral co-infection. In another embodiment, the level of TRAIL is used to distinguish between a viral and bacterial/viral co-infection.

Thus, the level of TRAIL can be used to rule in a bacterial infection, rule in a viral infection, rule in a bacterial/viral infection or rule in a non-infectious state.

Once a bacterial infection or mixed bacterial/viral infection has been ruled in, the subject may be treated with an antibiotic.

Examples of antibiotic agents include, but are not limited to Daptomycin; Gemifloxacin; Telavancin; Ceftaroline; Fidaxomicin; Amoxicillin; Ampicillin; Bacampicillin; Carbenicillin; Cloxacillin; Dicloxacillin; Flucloxacillin; Mezlocillin; Nafcillin; Oxacillin; Penicillin G; Penicillin V; Piperacillin; Pivampicillin; Pivmecillinam; Ticarcillin; Aztreonam; Imipenem; Doripenem; Meropenem; Ertapenem; Clindamycin; Lincomycin; Pristinamycin; Quinupristin; Cefacetrile (cephacetrile); Cefadroxil (cefadroxyl); Cefalexin (cephalexin); Cefaloglycin (cephaloglycin); Cefalonium (cephalonium); Cefaloridine (cephaloradine); Cefalotin (cephalothin); Cefapirin (cephapirin); Cefatrizine; Cefazaflur; Cefazedone; Cefazolin (cephazolin); Cefradine (cephradine); Cefroxadine; Ceftezole; Cefaclor; Cefamandole; Cefmetazole; Cefonicid; Cefotetan; Cefoxitin; Cefprozil (cefproxil); Cefuroxime; Cefuzonam; Cefcapene; Cefdaloxime; Cefdinir; Cefditoren; Cefetamet; Cefixime; Cefmenoxime; Cefodizime; Cefotaxime; Cefpimizole; Cefpodoxime; Cefteram; Ceftibuten; Ceftiofur; Ceftiolene; Ceftizoxime; Ceftriaxone; Cefoperazone; Ceftazidime; Cefclidine; Cefepime; Cefluprenam; Cefoselis; Cefozopran; Cefpirome; Cefquinome; Fifth Generation; Ceftobiprole; Ceftaroline; Not Classified; Cefaclomezine; Cefaloram; Cefaparole; Cefcanel; Cefedrolor; Cefempidone; Cefetrizole; Cefivitril; Cefmatilen; Cefmepidium; Cefovecin; Cefoxazole; Cefrotil; Cefsumide; Cefuracetime; Ceftioxide; Azithromycin; Erythromycin; Clarithromycin; Dirithromycin; Roxithromycin; Telithromycin; Amikacin; Gentamicin; Kanamycin; Neomycin; Netilmicin; Paromomycin; Streptomycin; Tobramycin; Flumequine; Nalidixic acid; Oxolinic acid; Piromidic acid; Pipemidic acid; Rosoxacin; Ciprofloxacin; Enoxacin; Lomefloxacin; Nadifloxacin; Norfloxacin; Ofloxacin; Pefloxacin; Rufloxacin; Balofloxacin; Gatifloxacin; Grepafloxacin; Levofloxacin; Moxifloxacin; Pazufloxacin; Sparfloxacin; Temafloxacin; Tosufloxacin; Besifloxacin; Clinafloxacin; Gemifloxacin; Sitafloxacin; Trovafloxacin; Prulifloxacin; Sulfamethizole; Sulfamethoxazole; Sulfisoxazole; Trimethoprim-Sulfamethoxazole; Demeclocycline; Doxycycline; Minocycline; Oxytetracycline; Tetracycline; Tigecycline; Chloramphenicol; Metronidazole; Tinidazole; Nitrofurantoin; Vancomycin; Teicoplanin; Telavancin; Linezolid; Cycloserine 2; Rifampin; Rifabutin; Rifapentine; Bacitracin; Polymyxin B; Viomycin; Capreomycin.

Once a viral infection has been ruled in, the subject may be treated with an anti-viral treatment.

An “anti-viral treatment” includes the administration of a compound, drug, regimen or an action that when performed by a subject with a viral infection can contribute to the subject's recovery from the infection or to a relief from symptoms. Examples of antiviral agents include, but are not limited to Abacavir; Aciclovir; Acyclovir; Adefovir; Amantadine; Amprenavir; Ampligen; Arbidol; Atazanavir; Atripla; Balavir; Boceprevirertet; Cidofovir; Combivir; Dolutegravir; Darunavir; Delavirdine; Didanosine; Docosanol; Edoxudine; Efavirenz; Emtricitabine; Enfuvirtide; Entecavir; Ecoliever; Famciclovir; Fomivirsen; Fosamprenavir; Foscarnet; Fosfonet; Fusion inhibitor; Ganciclovir; Ibacitabine; Imunovir; Idoxuridine; Imiquimod; Indinavir; Inosine; Integrase inhibitor; Interferon type III; Interferon type II; Interferon type I; Interferon; Lamivudine; Lopinavir; Loviride; Maraviroc; Moroxydine; Methisazone; Nelfinavir; Nevirapine; Nexavir; Oseltamivir; Peginterferon alfa-2a; Penciclovir; Peramivir; Pleconaril; Podophyllotoxin; Raltegravir; Reverse transcriptase inhibitor; Ribavirin; Rimantadine; Ritonavir; Pyramidine; Saquinavir; Sofosbuvir; StavudineTelaprevir; Tenofovir; Tenofovir disoproxil; Tipranavir; Trifluridine; Trizivir; Tromantadine; Truvada; traporved; Valaciclovir; Valganciclovir; Vicriviroc; Vidarabine; Viramidine; Zalcitabine; Zanamivir; Zidovudine; RNAi antivirals; inhaled rhibovirons; monoclonal antibody respigams; neuriminidase blocking agents.

According to a particular embodiment, the level of TRAIL is used to rule in an acute infection (e.g. an acute bacterial infection).

An “Acute Infection” is characterized by rapid onset of disease, a relatively brief period of symptoms, and resolution within days.

In another embodiment, the level of TRAIL is used to rule in a chronic infection.

A “chronic infection” is an infection that develops slowly and lasts a long time. Viruses that may cause a chronic infection include Hepatitis C and HIV. One difference between acute and chronic infection is that during acute infection the immune system often produces IgM+ antibodies against the infectious agent, whereas the chronic phase of the infection is usually characteristic of IgM−/IgG+ antibodies. In addition, acute infections cause immune mediated necrotic processes while chronic infections often cause inflammatory mediated fibrotic processes and scaring (e.g. Hepatitis C in the liver). Thus, acute and chronic infections may elicit different underlying immunological mechanisms.

By infection type is meant to include bacterial infections, mixed infections, viral infections, no infection, infectious or non-infectious.

By “ruling in” an infection it is meant that the subject has that type of infection.

Furthermore, the level of TRAIL can be used to rule out a bacterial infection, rule out a viral infection, rule out a bacterial/viral infection or rule out a non-infectious state.

By “ruling out” an infection it is meant that the subject does not have that type of infection.

A threshold value for TRAIL which may be used to indicate a bacterial infection may be less than 100 pg/ml, less than 90 pg/ml, less than 80 pg/ml, less than 70 pg/ml, less than 60 pg/ml, less than 50 pg/ml, less than 40 pg/ml.

A threshold value for TRAIL which may be used to indicate a viral infection may be greater than 100 pg/ml, greater than 110 pg/ml, greater than 120 pg/ml, greater than 130 pg/ml, greater than 140 pg/ml, less than 150 pg/ml, greater than 160 pg/ml.

In one embodiment, the immunoassay is configured so that TRAIL can be classified as being low (<70 pg/ml), medium (70-100 pg/ml) or high (>100 pg/ml).

Classification of subjects into subgroups (e.g. bacterially infected/viral infected; infectious/non-infectious) according to this aspect of the present invention is preferably done with an acceptable level of clinical or diagnostic accuracy. An “acceptable degree of diagnostic accuracy”, is herein defined as a test or assay (such as the test used in some aspects of the invention) in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, 0.80, desirably at least 0.85, more desirably at least 0.875, preferably at least 0.90, more preferably at least 0.925, and most preferably at least 0.95.

Alternatively, the methods can be used to diagnose with at least 75% total accuracy, more preferably 80%, 85%, 90%, 95%, 97%, 98%, 99% or greater total accuracy. Alternatively, the methods predict the correct management or treatment with an MCC larger than 0.2, 0.3, 0.4, 0.5, 0.6, 0.7, 0.8, 0.9 or 1.0.

It will be appreciated that the TRAIL level may be used in conjunction with other markers/tests/disease associated parameters etc. in order to diagnose the patient.

Examples of polypeptides that may be measured together with TRAIL contemplated by the present inventors are those set forth in Table 3 herein below.

TABLE 3

| Protein | Full | RefSeq DNA | RefSeq |
|---|---|---|---|
| symbol | Gene Name | sequence | proteins |
| CRP | C-reactive protein, | NC_000001.11 | NP_000558.2 |
| | pentraxin-related | NT_004487.20 | |
| | | NC_018912.2 | |
| IP-10 | Chemokine | NC_000004.12 | NP_001556.2 |
| | (C-X-C motif) | NC_018915.2 | |
| | ligand 10 | NT_016354.20 | |
| IL1R/ | Interleukin 1 | NC_000002.12 | NP_000868.1 |
| IL1R1/ | receptor, | NT_005403.18 | NP_001275635.1 |
| IL1RA | type I | NC_018913.2 | |
| Procalcitonin | Calcitonin-related | NC_000011.10 | NP_001029124.1 |
| (PCT) | polypeptide alpha | NC_018922.2 | NP_001029125.1 |
| | | NT_009237.19 | NP_001732.1 |
| SAA/SAA1 | Serum | NC_000011.10 | NP_000322.2 |
| | amyloid A1 | NC_018922.2 | NP_001171477.1 |
| | | NT_009237.19 | NP_954630.1 |
| TREM1 | Triggering receptor | NC_000006.12 | NP_001229518.1 |
| | expressed on | NT_007592.16 | NP_001229519.1 |
| | myeloid cells 1 | NC_018917.2 | NP_061113.1 |
| TREM2 | Triggering receptor | NC_000006.12 | NP_001258750.1 |
| | expressed on | NT_007592.16 | NP_061838.1 |
| | myeloid cells 2 | NC_018917.2 | |
| RSAD2 | Radical S-adenosyl | NC_000002.12 | NP_542388.2 |
| | methionine domain | NT_005334.17 | |
| | containing 2 | NC_018913.2 | |
| NGAL | Lipocalin 2 | NC_000009.12 | NP_005555.2 |
| | | NC_018920.2 | |
| | | NT_008470.20 | |
| MMP8 | Matrix | NC_000011.10 | NP_001291370.1 |
| | metallopeptidase 8 | NT_033899.9 | NP_001291371.1 |
| | | NC_018922.2 | NP_002415.1 |
| MX1 | MX Dynamin-Like | NC_000021.9 | NP_001138397.1 |
| | GTPase | NT_011512.12 | NP_001171517.1 |
| | 1 | NC_018932.2 | NP_001269849.1 |
| | | | NP_002453.2 |

In one embodiment, at least one, at least two, at least three, at least four, at least five or all of the following markers are measured together with TRAIL-CRP, IP10, IL-6, NGAL, PCT and MX1.

Other examples of polypeptides that may be measured together with TRAIL using antibodies of this aspect of the present invention include but are not limited to: OTOF, PI3, CYBRD1, EIF2AK2, CMPK2, IL1RA, IP10, Mac-2BP, B2M, BCA-1, CHI3L1, Eotaxin, IL1a, MCP, CD62L, VEGFR2, CHP, CMPK2, CORO1C, EIF2AK2, ISG15, RPL22L1, RTN3, CD112, CD134, CD182, CD231, CD235A, CD335, CD337, CD45, CD49D, CD66A/C/D/E, CD73, CD84, EGFR, GPR162, HLA-A/B/C, ITGAM, NRG1, RAP1B, SELI, SPINT2, SSEA1, IL1, I-TAC, TNFR1, IFITM3, IFIT3, EIF4B, IFIT1, LOC26010, MBOAT2, MX1, OAS2, RSAD2, ADIPOR1, CD15, CD8A, IFITM1, IL7, CRP, SAA, TREM-1, PCT, IL-8, TREM-1, IL6, ARG1, ARPC2, ATP6V0B, BCA-1, BRI3BP, CCL19-MIP3b, CES1, CORO1A, HERC5, IFI6, IFIT3, KIAA0082, LIPT1, LRDD, MCP-2, PARP9, PTEN, QARS, RAB13, RPL34, SART3, TRIM22, UBE2N, XAF1 and ZBP1.

IL1RA: The protein encoded by this gene is a cytokine receptor that belongs to the interleukin 1 receptor family. Additional names of the gene include without limitations: CD121A, IL-1RT1, p80, CD121a antigen, CD121A, IL1R and IL1RA.

A representative RefSeq amino acid sequence of this protein is NP_000558.2. Representative RefSeq DNA sequences include: NC_000001.11, NT_004487.20, NC_018912.2.

PCT: Procalcitonin (PCT) is a peptide precursor of the hormone calcitonin

A representative RefSeq amino acid sequence of this protein is NP_000558.2. Representative RefSeq DNA sequences include: NC_000001.11, NT_004487.20, NC_018912.2.

SAA: encodes a member of the serum amyloid A family of apolipoproteins.

A representative RefSeq amino acid sequence of this protein is NP_000558.2. Representative RefSeq DNA sequences include: NC_000001.11, NT_004487.20, NC_018912.2.

CHP: A representative RefSeq amino acid sequence of this protein is NP_009167.1. Representative RefSeq DNA sequences include: NC_000015.10, NT_010194.18, NC_018926.2.

CMPK2: A representative RefSeq amino acid sequence of this protein is NP_001243406.1, NP_001243407.1, NP_997198.2. Representative RefSeq DNA sequences include: NC_000002.12, NT_005334.17, NC_018913.2.

CORO1C: A representative RefSeq amino acid sequence of this protein is NP_001098707.1, NP_001263400.1, NP_055140.1. Representative RefSeq DNA sequences include: NC_000012.12, NT_029419.13, NC_018923.2.

EIF2AK2: Additional aliases include without limitation: PKR, PRKR, EIF2AK1, protein kinase, interferon-inducible double stranded RNA dependent, p68 kinase.

A representative RefSeq amino acid sequence of this protein is NP_001129123.1, NP_001129124.1, NP_002750.1. Representative RefSeq DNA sequences include: NC_000002.12, NT_022184.16, NC_018913.2.

ISG15: ISG15 ubiquitin-like modifier; additional aliases of ISG15 include without limitation G1P2, IFI15, IP17, UCRP and hUCRP.

A representative RefSeq amino acid sequence of this protein is NP_005092.1. Representative RefSeq DNA sequences include: NC_000001.11, NC_018912.2, NT_032977.10.

RTN3: A representative RefSeq amino acid sequence of this protein is NP_001252518.1, NP_001252519.1, NP_001252520.1, NP_006045.1, NP_958831.1, NP_958832.1, NP_958833.1. Representative RefSeq DNA sequences include: NC_000011.10, NT_167190.2, NC_018922.2.

CD112: This gene encodes a single-pass type I membrane glycoprotein with two Ig-like C2-type domains and an Ig-like V-type domain.

A representative RefSeq amino acid sequence of this protein is NP_001036189.1, NP_002847.1. Representative RefSeq DNA sequences include: NC_000019.10, NT_011109.17, NC_018930.2.

CD134: The protein encoded by this gene is a member of the TNF-receptor superfamily.

A representative RefSeq amino acid sequence of this protein is NP_003318.1. Representative RefSeq DNA sequences include: NC_000001.11, NT_004487.20, NC_018912.2.

CD182: The protein encoded by this gene is a member of the G-protein-coupled receptor family.

A representative RefSeq amino acid sequence of this protein is NP_001136269.1 or NP_001495.1. Representative RefSeq DNA sequences include: NC_000023.11, NT_011651.18, NC_018934.2.

CD231: The protein encoded by this gene is a member of the transmembrane 4 superfamily, also known as the tetraspanin family.

A representative RefSeq amino acid sequence of this protein is Representative RefSeq DNA sequences include: NC_000023.11, NC_018934.2, NT_079573.5.

CD235a: CD235a is the major intrinsic membrane protein of the erythrocyte.

A representative RefSeq amino acid sequence of this protein is NP_002090.4. Representative RefSeq DNA sequences include: NC_000004.12, NT_016354.20, NC_018915.2.

CD335: Representative RefSeq amino acid sequence of this protein are NP_001138929.2, NP_001138930.2, NP_001229285.1, NP_001229286.1 or NP_004820.2. Representative RefSeq DNA sequences include: NC_000019.10, NT_011109.17, NT_187693.1, NC_018930.2, NT_187671.1, NT_187674.1, NT_187675.1, NT_187676.1, NT_187677.1, NT_187683.

CD337: The protein encoded by this gene is a natural cytotoxicity receptor (NCR).

Representative RefSeq amino acid sequences of this protein are NP_001138938.1, NP_001138939.1, NP_667341.1. Representative RefSeq DNA sequences include: NC_000006.12, NT_007592.16, NT_167244.2, NT_113891.3, NT_167245.2, NT_167246.2, NT_167247.2, NT_167248.2, NT_167249.2, NC_018917.2.

CD45: The protein encoded by this gene is a member of the protein tyrosine phosphatase (PTP) family.

Representative RefSeq amino acid sequences of this protein are NP_001254727.1, NP_002829.3, NP_563578.2. Representative RefSeq DNA sequences include: NC_000001.11, NT_004487.20, NC_018912.2.

CD49d: The product of this gene belongs to the integrin alpha chain family of proteins.

A representative RefSeq amino acid sequences of this protein is NP_000876.3. Representative RefSeq DNA sequences include: NC_000002.12, NC_018913.2, NT_005403.18.

CD66a: This gene encodes a member of the carcinoembryonic antigen (CEA) gene family, which belongs to the immunoglobulin superfamily.

Representative RefSeq amino acid sequences of this protein are NP_001020083.1, NP_001171742.1, NP_001171744.1, NP_001171745.1, NP_001192273.1, NP_001703.2. Representative RefSeq DNA sequences include: NC_000019.10, NT_011109.17, NC_018930.2.

CD66c: Carcinoembryonic antigen (CEA; MIM 114890).

A representative RefSeq amino acid sequences of this protein is NP_002474.4. Representative RefSeq DNA sequences include: NC_000019.10, NT_011109.17, NC_018930.2.

CD66d: This gene encodes a member of the family of carcinoembryonic antigen-related cell adhesion molecules (CEACAMs).

Representative RefSeq amino acid sequences of this protein are NP_001264092.1, NP_001806.2. Representative RefSeq DNA sequences include: NC_000019.10, NC_018930.2, NT_011109.17.

CD66e: CD66e is a member of the CEACAM subfamily.

Representative RefSeq amino acid sequences of this protein are NP_001278413.1, NP_004354.3. Representative RefSeq DNA sequences include: NC_000019.10, NT_011109.17, NC_018930.2.

CD84: Representative RefSeq amino acid sequences of this protein are NP_001171808.1, NP_001171810.1, NP_001171811.1, NP_003865.1. Representative RefSeq DNA sequences include: NC_000001.11, NT_004487.20, NC_018912.2.

EGFR: The protein encoded by this gene is a transmembrane glycoprotein that is a member of the protein kinase superfamily.

Representative RefSeq amino acid sequences of this protein are NP_005219.2, NP_958439.1, NP_958440.1, NP_958441.1. Representative RefSeq DNA sequences include: NC_000007.14, NC_018918.2, NT_007819.18.

GPR162: Representative RefSeq amino acid sequences of this protein are NP_055264.1 or NP_062832. Representative RefSeq DNA sequences include: NC_000012.12, NC_018923.2, NT_009759.17.

HLA-A: HLA-A belongs to the HLA class I heavy chain paralogues.

Representative RefSeq amino acid sequences of this protein are NP_001229687.1 or NP_002107.3. Representative RefSeq DNA sequences include: NT_167247.2, NC_018917.2, NT_113891.3, NT_167244.2, NC_000006.12, NT_007592.16, NT_167245.2, NT_167246.2, NT_167248.2, NT_167249.2.

HLA-B: HLA-B belongs to the HLA class I heavy chain paralogues.

A representative RefSeq amino acid sequence of this protein is NP_005505.2. Representative RefSeq DNA sequences include: NT_167246.2, NT_167249.2, NT_167247.2, NC_000006.12, NT_007592.16, NT_113891.3, NT_167248.2, NC_018917.2.

HLA-C: HLA-C belongs to the HLA class I heavy chain paralogues.

Representative RefSeq amino acid sequences of this protein are NP_001229971.1, NP_002108.4. Representative RefSeq DNA sequences include: NT_113891.3, NC_000006.12, NC_018917.2, NT_007592.16, NT 167245.2, NT_167246.2, NT_167247.2, NT_167248.2, NT_167249.2.

ITGAM: This gene encodes the integrin alpha M chain.

Representative RefSeq amino acid sequences of this protein are NP_000623.2, NP_001139280.1. Representative RefSeq DNA sequences include: NC_000016.10, NT_187260.1, NC_018927.2.

NRG1: Representative RefSeq amino acid sequences of this protein are of NP_001153467.1, NP_001153468.1, NP_001153471.1, NP_001153473.1, NP_001153474.1, NP_001153476.1, NP_001153477.1, NP_001153479.1, NP_001153480.1, NP_004486.2, NP_039250.2, NP_039251.2, NP_039252.2, NP_039253.1, NP_039254.1, NP_039256.2, NP_039258.1. Representative RefSeq DNA sequences include: NC_000008.11, NT_167187.2, NC_018919.2.

RAPB: GTP-binding protein that possesses intrinsic GTPase activity. Representative RefSeq amino acid sequences of this protein are NP_001010942.1, NP_001238846.1, NP_001238847.1, NP_001238850.1, NP_001238851.1, NP_056461.1. Representative RefSeq DNA sequences include: NC_000012.12, NC_018923.2, NT_029419.13.

SELI: This gene encodes a selenoprotein, which contains a selenocysteine (Sec) residue at its active site. A representative RefSeq amino acid sequence of this protein is NP_277040.1. Representative RefSeq DNA sequences include: NC_000002.12, NC_018913.2, NT_022184.16.

SPINT2: This gene encodes a transmembrane protein with two extracellular Kunitz domains Representative RefSeq amino acid sequences of this protein are NP_001159575.1 or NP_066925.1. Representative RefSeq DNA sequences include: NC_000019.10, NC_018930.2, NT_011109.17.

EIF4B: Representative RefSeq amino acid sequences of this protein are NP_001287750.1 or NP_001408.2. Representative RefSeq DNA sequences include: NC_000012.12, NT_029419.13, NC_018923.2.

IFIT1: Interferon-induced protein with tetratricopeptide repeats. Representative RefSeq amino acid sequences of this protein are NP_001257856.1, NP_001257857.1, NP_001257858.1, NP_001257859.1, NP_001539.3. Representative RefSeq DNA sequences include: NC_000010.11, NC_018921.2, NT_030059.14.

IFITM3/IFITM2: IFN-induced antiviral protein. A representative RefSeq amino acid sequence of this protein is NP_066362.2. Representative RefSeq DNA sequences include: NC 000011.10. NC_018922.2, NT_009237.19.

RSAD2: Radical S-adenosyl methionine domain containing 2; additional aliases of RSAD2 include without limitation 2510004L01Rik, cig33, cig5 and vig1. Representative RefSeq amino acid sequences of this protein are NP_001277482.1, NP_001277486.1, NP_001277558.1, NP_057083.2. Representative RefSeq DNA sequences include: NC_000002.12, NT_005334.17, NC_018913.2.

ADIPOR1: ADIPOR1 is a receptor for globular and full-length adiponectin (APM1). Representative RefSeq amino acid sequences of this protein are NP_001277482.1, NP_001277486.1, NP_001277558.1, NP_057083.2. Representative RefSeq DNA sequences include: NC_000001.11, NC_018912.2, NT_004487.20.

CD15 (FUT4): A representative RefSeq amino acid sequence of this protein is NP_002024.1. Representative RefSeq DNA sequences include: NC_000011.10, NC_018922.2, NT_033899.9.

CD73: Representative RefSeq amino acid sequences of this protein are NP_001191742.1 or NP_002517.1. Representative RefSeq DNA sequences include: NC_000006.12, NC_018917.2, NT_025741.16.

CD8A: The CD8 antigen is a cell surface glycoprotein. Representative RefSeq amino acid sequences of this protein are NP_001139345.1, NP_001759.3 or NP_741969.1. Representative RefSeq DNA sequences include: NC_000002.12, NC_018913.2, NT_022184.16.

IFITM1: Encodes an IFN-induced antiviral protein. A representative RefSeq amino acid sequence of this protein is NP_003632.3. Representative RefSeq DNA sequences include: NC_000011.10, NC_018922.2, NT_009237.19.

IFITM3: Encodes an IFN-induced antiviral protein. A representative RefSeq amino acid sequence of this protein is NP_066362.2. Representative RefSeq DNA sequences include: NC_000011.10, NC_018922.2, NT_009237.19.

IL7R: The protein encoded by this gene is a receptor for interleukine 7 (IL7). A representative RefSeq amino acid sequence of this protein is NP_002176.2. Representative RefSeq DNA sequences include: NC_000005.10, NT_006576.17, NC_018916.2.

LOC26010 (SPATS2L DNAPTP6): Representative RefSeq amino acid sequences of this protein are NP_001093892.1, NP_001093893.1, NP_001093894.1, NP_001269664.1, NP_001269672.1, NP_001269673.1, NP_056350.2. RefSeq DNA sequence: NC_000002.12, NT_005403.18, NC_018913.2.

TREM1: Triggering receptor expressed on myeloid cells 1; additional aliases of TREM1 are CD354 and TREM-1. Representative RefSeq amino acid sequences of this protein are NP_001229518.1, NP_001229519.1, NP_061113.1. Representative RefSeq DNA sequences include: NC_000001.11, NT_004487.20, NC_018912.2.

IL6: A representative RefSeq amino acid sequences of this protein is NP_000591.1. Representative RefSeq DNA sequences include: NC_000007.14, NT_007819.18, NC_018918.2.

IL7: This gene encodes a cytokine. Representative RefSeq amino acid sequences of this protein are NP_000871.1, NP_001186815.1, NP_001186816.1, NP_001186817.1. Representative RefSeq DNA sequences include: NC_000008.11, NT_008183.20, NC_018919.2.

ARG1: Arginase. Representative RefSeq amino acid sequences of this protein are NP_000036.2 or NP_001231367.1. Representative RefSeq DNA sequences include: NC_000006.12, NT_025741.16, NC_018917.2.

ARPC2: This gene encodes one of seven subunits of the human Arp2/3 protein complex. Representative RefSeq amino acid sequences of this protein are NP_005722.1 or NP_690601.1. Representative RefSeq DNA sequences include: NC_000002.12, NT_005403.18, NC_018913.2.

ATP6V0B: H+-ATPase (vacuolar ATPase, V-ATPase) is an enzyme transporter. Representative RefSeq amino acid sequences of this protein are NP_001034546.1, NP_001281262.1 or NP_004038.1. Representative RefSeq DNA sequences include: NC_000001.11, NC_018912.2, NT_032977.10.

BRI3BP: A representative RefSeq amino acid sequence of this protein is NP_542193.3. Representative RefSeq DNA sequences include: NC_000012.12, NT_029419.13, NC_018923.2.

CCL19: A representative RefSeq amino acid sequence of this protein is NP_006265.1. Representative RefSeq DNA sequences include: NC_000009.12, NC_018920.2, NT_008413.19.

CES1: Representative RefSeq amino acid sequences of this protein are NP_001020365.1, NP_001020366.1 or NP_001257.4. Representative RefSeq DNA sequences include: NC_000016.10, NT_010498.16, NC_018927.2.

CORO1A: Representative RefSeq amino acid sequences of this protein are NP_001180262.1 or NP_009005.1. Representative RefSeq DNA sequences include: NC_000016.10, NT_187260.1, NC_018927.2.

HERC5: A representative RefSeq amino acid sequence of this protein is NP_057407.2. Representative RefSeq DNA sequences include: NC_000004.12, NT_016354.20, NC_018915.2.

IFI6: Representative RefSeq amino acid sequences of this protein are NP_002029.3, NP_075010.1, NP_075011.1. Representative RefSeq DNA sequences include: NC_000001.11, NC_018912.2, NT_032977.10.

IFIT3: Additional aliases of the protein include without limitation: interferon-induced protein with tetratricopeptide repeats 3, IFI60, ISG60 and Interferon-induced 60 kDa protein.

Representative RefSeq amino acid sequences of this protein are NP_001026853.1, NP_001276687.1, NP_001276688.1, NP_001540.2. Representative RefSeq DNA sequences include: NC_000010.11, NC_018921.2, NT_030059.14.

MBOAT2: Acyltransferase. A representative RefSeq amino acid sequence of this protein is NP_620154.2. Representative RefSeq DNA sequences include: NC_000002.12, NT_005334.17, NC_018913.2.

MX1/MXA: myxovirus (influenza virus) resistance 1; additional aliases of MX1 include without limitation IFI-78K, IFI78, MX and MxA. Representative RefSeq amino acid sequences of this protein are NP_001138397.1, NP_001171517.1, NP_001269849.1, NP_002453.2. Representative RefSeq DNA sequences include: NC_000021.9, NT_011512.12, NC_018932.2.

OAS2: This gene encodes a member of the 2-5A synthetase family. Representative RefSeq amino acid sequences of this protein are NP_001027903.1, NP_002526.2, NP_058197.2. Representative RefSeq DNA sequences include: NC_000012.12, NT_029419.13, NC_018923.2.

KIAA0082 (FTSJD2): S-adenosyl-L-methionine-dependent methyltransferase. A representative RefSeq amino acid sequence of this protein is NP_055865.1. Representative RefSeq DNA sequences include: NC_000006.12, NT_007592.16, NC_018917.2.

LIPT1: Representative RefSeq amino acid sequences of this protein are NP_001191759.1, NP_057013.1, NP_660198.1, NP_660199.1, NP_660200.1. Representative RefSeq DNA sequences include: NC_000002.12, NC_018913.2, NT_005403.18.

LRDD: Representative RefSeq amino acid sequences of this protein are NP_665893.2 or NP_665894.2. Representative RefSeq DNA sequences include: NC_000011.10, NT_009237.19, NC_018922.2.

MCP-2: This gene encodes a cytokine. A representative RefSeq amino acid sequence of this protein is NP_005614.2. Representative RefSeq DNA sequences include: NC_000017.11, NC_018928.2, NT_010783.16.

PARP9: Poly (ADP-ribose) polymerase (PARP). Representative RefSeq amino acid sequences of this protein are NP_001139574.1, NP_001139575.1, NP_001139576.1, NP_001139577.1, NP_001139578.1, NP_113646.2. Representative RefSeq DNA sequences include: NC_000003.12, NT_005612.17, NC_018914.2.

PTEN: Representative RefSeq amino acid sequences of this protein are NP_000305.3, NP_001291646.2, NP_001291647.1. Representative RefSeq DNA sequences include: NC_000010.11, NT_030059.14, NC_018921.2.

OARS: Aminoacyl-tRNA synthetases catalyze the aminoacylation of tRNA by their cognate amino acid. Representative RefSeq amino acid sequences of this protein are NP_001259002.1 or NP_005042.1. Representative RefSeq DNA sequences include: NC_000003.12, NT_022517.19, NC_018914.2.

RAB13: Representative RefSeq amino acid sequences of this protein are NP_001258967.1, NP_002861.1. Representative RefSeq DNA sequences include: NC_000001.11, NC_018912.2, NT_004487.20.

RPL22L1: A representative RefSeq amino acid sequence of this protein is NP_001093115.1. Representative RefSeq DNA sequences include: NC_000003.12, NT_005612.17, NC_018914.2.

RPL34: The protein belongs to the L34E family of ribosomal proteins. Representative RefSeq amino acid sequences of this protein are NP_000986.2 or NP_296374.1. Representative RefSeq DNA sequences include: NC_000004.12, NT_016354.20, NC_018915.2.

SART3: The protein encoded by this gene is an RNA-binding nuclear protein. A representative RefSeq amino acid sequence of this protein is NP_055521.1. Representative RefSeq DNA sequences include: NC_000012.12, NT_029419.13, NC_018923.2.

SSEA-1: A representative RefSeq amino acid sequence of this protein is NP_002024.1. Representative RefSeq DNA sequences include: NC_000011.10, NC_018922.2, NT_033899.9.

TRIM22: Interferon-induced antiviral protein. Representative RefSeq amino acid sequences of this protein are NP_001186502.1 or NP_006065.2. Representative RefSeq DNA sequences include: NC_000011.10, NC_018922.2, NT_009237.19.

UBE2N: A representative RefSeq amino acid sequence of this protein is NP_003339.1. Representative RefSeq DNA sequences include: NC_000012.12, NT_029419.13, NC_018923.2.

XAF1: Representative RefSeq amino acid sequences of this protein are NP_059993.2 or NP_954590.1. Representative RefSeq DNA sequences include: NC_000017.11, NT_010718.17, NC_018928.2.

ZBP1: Representative RefSeq amino acid sequences of this protein are NP_001082.2 or NP_001259001.1. Representative RefSeq DNA sequences include: NC_000007.14, NT_007933.16, NC_018918.2.

IL11: The protein encoded by this gene is a member of the gp130 family of cytokines. Representative RefSeq amino acid sequences of this protein are NP_000632.1 or NP_001254647.1. Representative RefSeq DNA sequences include: NC_000019.10, NC_018930.2, NT_011109.17.

I-TAC: Additional names of the gene include without limitations: SCYB11, SCYB9B and CXCL11. Representative RefSeq amino acid sequences of this protein are NP_001289052.1, NP_005400.1. Representative RefSeq DNA sequences include: NC_000004.12, NC_018915.2, NT_016354.20.

TNFR1: Receptor for TNFSF2/TNF-alpha and homotrimeric TNFSF1/lymphotoxin-alpha. Additional names of the gene include without limitations: TNFRSF1A, TNFAR, p55, p60, CD120a antigen and CD120a antigen.

A representative RefSeq amino acid sequence of this protein is NP_003780.1. Representative RefSeq DNA sequences include: NC_000016.10, NT_010498.16, NC_018927.2.

IL-8: The protein encoded by this gene is a member of the CXC chemokine family. Additional aliases of IL-8 include without limitation: Interleukin 8, K60, CXCL8, SCYB8, GCP-1, TSG-1, MDNCF, b-ENAP, MONAP, alveolar macrophage chemotactic factor I, NAP-1, beta endothelial cell-derived neutrophil activating peptide, GCP1, beta-thromboglobulin-like protein, LECT, chemokine (C—X—C motif) ligand 8, LUCT, emoctakin, LYNAP, interleukin-8, NAF, lung giant cell carcinoma-derived chemotactic protein, NAP1, lymphocyte derived neutrophil activating peptide, IL-8, neutrophil-activating peptide 1, Granulocyte chemotactic protein 1, small inducible cytokine subfamily B, member 8, Monocyte-derived neutrophil chemotactic factor, tumor necrosis factor-induced gene 1, Monocyte-derived neutrophil-activating peptide, Emoctakin, T-cell chemotactic factor, C—X—C motif chemokine 8, 3-10C,Neutrophil-activating protein 1, AMCF-I and Protein 3-10C.

A representative RefSeq amino acid sequence of this protein is NP_000575.1. Representative RefSeq DNA sequences include: NC_000004.12, NC_018915.2, NT_016354.20.

HP—This gene encodes a preproprotein, which is processed to yield both alpha and beta chains, which subsequently combine as a tetramer to produce haptoglobin. Representative RefSeq amino acid sequences of this protein are NP_001119574.1, or NP_005134.1. Representative RefSeq DNA sequences include: NC_000016.10, NT_010498.16, NC_018927.2.

Other contemplated uses of the TRAIL antibodies of the present invention include determining the severity of a disease (including both infectious and non-infectious), providing risk assessments of pre-diagnosed diseases (including both infectious and non-infectious) and determining a management course of a pre-diagnosed disease (including both infectious and non-infectious).

Such diseases include for example serious bacterial diseases such as meningitis, sepsis, pneumonia, septic arthritis and cellulitis, bacteremia, urinary tract infection (UTI), Pyelonephritis, Meningococcal Disease, Invasive; Staphylococcus Aureus Infections; Drug resistant (MRSA, VISA, VRSA) Streptococcal Disease, Group A Invasive or Streptococcal TSS and Streptococcal Disease.

In another embodiment, the level of TRAIL is used to determine a management course for treating a subject having a disease, for example when the TRAIL level is below a predetermined level, the subject is treated as a high-risk patient. In another embodiment, the level of TRAIL is used for determining prognosis of disease and treatment thereof.

As mentioned above, when the TRAIL protein serum level is lower than a predetermined level, the subject is classified as a high-risk patient. The predetermined level may be below 30 pg/ml, below 25 pg/ml, below 20 pg/ml, below 15 pg/ml or even below 10 pg/ml.

Traditional risk factors and additional clinical parameters may be measured together with TRAIL for such purposes and also for distinguishing between bacterial and viral infections.

“Traditional laboratory risk factors” encompass biomarkers isolated or derived from subject samples and which are currently evaluated in the clinical laboratory and used in traditional global risk assessment algorithms, such as absolute neutrophil count (abbreviated ANC), absolute lymphocyte count (abbreviated ALC), white blood count (abbreviated WBC), neutrophil % (defined as the fraction of white blood cells that are neutrophils and abbreviated Neu (%)), lymphocyte % (defined as the fraction of white blood cells that are lymphocytes and abbreviated Lym (%)), monocyte % (defined as the fraction of white blood cells that are monocytes and abbreviated Mon (%)), Sodium (abbreviated Na), Potassium (abbreviated K), Bilirubin (abbreviated Bili).

“Clinical parameters” encompass all non-sample or non-analyte biomarkers of subject health status or other characteristics, such as, without limitation, age (Age), ethnicity (RACE), gender (Sex), core body temperature (abbreviated “temperature”), maximal core body temperature since initial appearance of symptoms (abbreviated “maximal temperature”), time from initial appearance of symptoms (abbreviated “time from symptoms”) or family history (abbreviated FamHX).

Other exemplary blood biomarkers which may be measured according to this aspect of the present invention include but are not limited to creatin, serum albumin, and interleukin-6.

Additional combinations of markers which may be used for risk management, treatment course and/or distinguishing between viral and bacterial infections include but are not limited to:

TRAIL+CRP; TRAIL+CRP+IP10; TRAIL+PCT; TRAIL+IL-6; TRAIL+IP-10; TRAIL+NGAL; TRAIL+CRP+PCT; TRAIL+CRP+NGAL; TRAIL+CRP+IP-10; TRAIL+CRP+IL-6; TRAIL+PCT+IL-6; TRAIL+PCT+IP-10; TRAIL+PCT+NGAL; TRAIL+CRP+IL-6+PCT; TRAIL+CRP+IL-6+NGAL; TRAIL+CRP+IL-6+IP-10; TRAIL+NGAL+IL-6+PCT; TRAIL+IP-10+IL-6+PCT; TRAIL+neopterin; TRAIL+WBC; TRAIL+ANC; TRAIL+temperature; TRAIL+mean arterial pressure; TRAIL+pH arterial; TRAIL+heart rate; TRAIL+respiratory rate; TRAIL+AaDO2 or PaO2; TRAIL+sodium; TRAIL+potassium; TRAIL+creatinine; TRAIL+hematocrit; TRAIL+MX1+CRP; TRAIL+MX1+RSAD2; TRAIL+CRP+RSAD2.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Methods

Epitope mapping was performed for three antibodies against Tumor necrosis factor ligand superfamily member 10 (TRAIL, TNFLSF10, NP_003801.1) which were found useful in measuring TRAIL for the differentiation between patients with bacterial and viral infections.

High resolution linear epitope mapping of TRAIL antibodies was performed using the PEPperCHIP™ Peptide Microarrays (by PEPperPRINT, Heidelberg, Germany) that enabled the high resolution epitope mapping of antibodies and sera by translating one or more antigens into overlapping peptides. Incubation of the resulting peptide microarray with an antibody or serum sample as well as suited secondary antibodies gives rise to spot patterns that correlate with the epitope of the given sample.

TRAIL amino acid sequence (SEQ ID NO: 96−MAMMEVQGGPSLGQTCVLIVIFTVLLQSLCVAVTYVYFTNELKQMQDKYSKSGIACFL KEDDSYWDPNDEESMNSPCWQVKWQLRQLVRKMILRTSEETISTVQEKQQNISPLVRE RGPQRVAAHITGTRGRSNTLSSPNSKNEKALGRKINSWESSRSGHSFLSNLHLRNGELVI HEKGFYYIYSQTYFRFQEEIKENTKNDKQMVQYIYKYTSYPDPILLMKSARNSCWSKDA EYGLYSIYQGGIFELKENDRIFVSVTNEHLIDMDHEASFFGAFLVG), was elongated by neutral GSGSGSG (SEQ ID NO: 99) linkers at the C- and N-terminus to avoid truncated peptides. The elongated antigen sequence was translated into 7, 10 and 13 amino acid peptides with peptide-peptide overlaps of 6, 9 and 12 amino acids for maximal epitope resolution.

After peptide synthesis, all peptides were cyclized via a thioether linkage between a C-terminal cysteine side chain and an appropriately modified N-terminus. The resulting conformational TRAIL peptide microarrays contained 858 different cyclic constrained peptides printed in triplicate (2,574 peptide spots), and were framed by additional hemagglutinin (HA) (YPYDVPDYAG (SEQ ID NO: 100; 114 spots) control peptides.

Pre-staining of two conformational TRAIL peptide microarray copies was done with either secondary goat anti-mouse IgG (H+L) DyLight680 antibody (1:5000) or with secondary donkey anti-goat IgG (H+L) DyLight680 antibody (1:5000) and control antibody mouse monoclonal anti-HA (12CA5) DyLight800 (1:2000) to investigate background interactions with the cyclic constrained antigen-derived peptides that could interfere with the main assays. Subsequent incubation of other TRAIL peptide microarrays with the antibody samples at concentrations of 1 μg/ml, 10 μg/ml, 100 μg/ml and 250 μg/ml in incubation buffer was followed by staining with secondary and control antibodies as well as read-out at scanning intensities of 6/7 or 7/7 (red/green). The additional HA peptides framing the peptide microarrays were simultaneously (goat antibody) or subsequently (mouse antibodies) stained as internal quality control to confirm the assay quality and the peptide microarray integrity.

Quantification of spot intensities and peptide annotation were based on the 16-bit gray scale tiff files at scanning intensities of 6/7 or 7/7 that exhibit a higher dynamic range than the 24-bit colorized tiff files. Microarray image analysis was done with PepSlide™ Analyzer. A software algorithm breaks down fluorescence intensities of each spot into raw, foreground and background signal, and calculates averaged median foreground intensities and spot-to-spot deviations of spot triplicates. Based on averaged median foreground intensities, an intensity map was generated and interactions in the peptide map highlighted by an intensity color code with red for high and white for low spot intensities. We tolerated a maximum spot-to-spot deviation of 40%, otherwise the corresponding intensity value was zeroed.

Averaged spot intensities of the assays with the antibody samples were plotted against the antigen sequence from the N- to the C-terminus of TRAIL to visualize overall spot intensities and signal-to-noise ratios. The intensity plots were correlated with peptide and intensity maps as well as with visual inspection of the microarray scans to identify epitopes that were recognized by the antibody samples.

Results

Goat Antibody Poly:

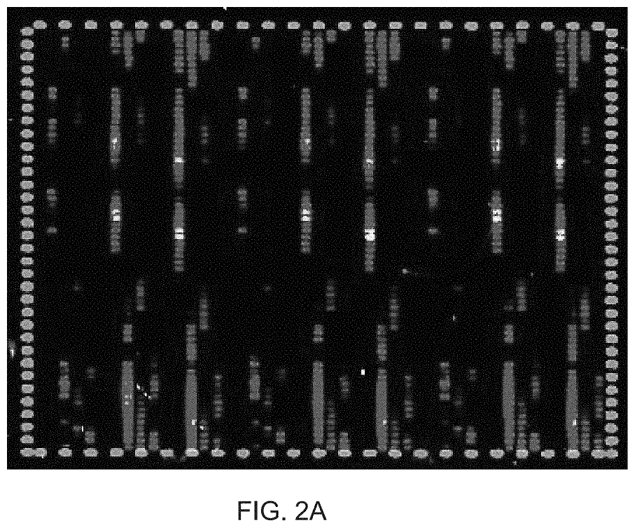

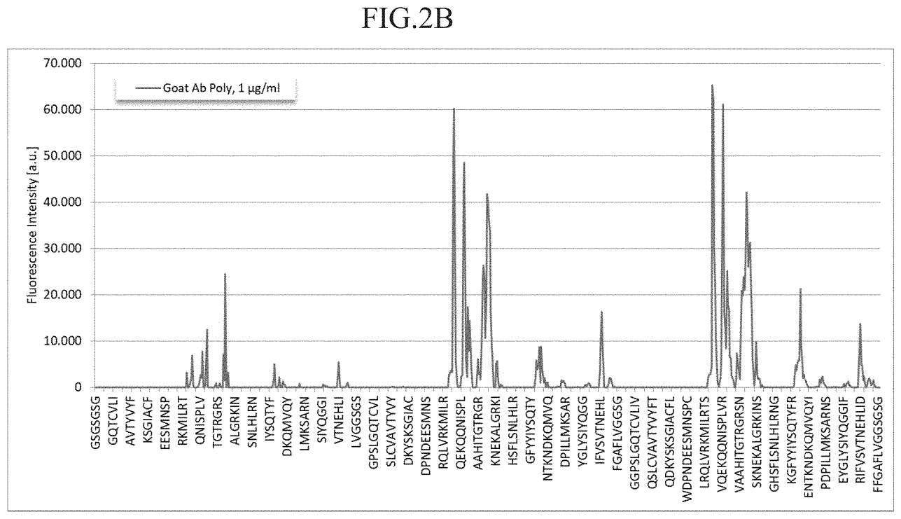

Incubation of goat antibody Poly at a concentration of 1 μg/ml was followed by staining with secondary and control antibodies as well as read out at scanning intensities of 6/7 (red/green; FIGS. 2A-B). A very strong antibody response against multiple epitope-like spot patterns were formed with the peptide motifs described in Table 4.

TABLE 4

| Epitope | |
|---|---|
| SEQ ID | |
| NO: which | |
| appear in | |
| order of | Epitope |
| the protein | sequence |
| 1 | STVQ |
| 2 | ISPLVRE |
| 3 | ERGPQR |
| 4 | GRSNTL |
| 5 | LSSPNSK |
| 6 | KALGRKIN |
| 7 | FRFQEE |
| 8 | IKENTK |
| 9 | VTNEHL |

The full-length sequences of
the epitopes are provide
in Table 5, herein below.

| Epitope | | |
|---|---|---|
| SEQ ID | | |
| NO: which | | |
| appear in | | |
| order of | | |
| the protein | Epitope | |
| (order) | sequence | Antibody |
| 101 | ETISTVQEK | Poly |
| 102 | NISPLVRE | Poly |
| 103 | ERGPQR | Poly |
| 104 | RGRSNTLS | Poly |
| 105 | LSSPNSK | Poly |
| 106 | KALGRKIN | Poly |
| 107 | FRFQEEI | Poly |
| 108 | IKENTK | Poly |
| 109 | SVTNEHLI | Poly |

Mouse Antibody Mono 2:

Incubation of mouse monoclonal antibody Mono 2 at concentrations of 10 μg/ml, 100 μg/ml (scans not shown) and 250 μg/ml was followed by staining with secondary goat anti-mouse IgG (H+L) DyLight680 antibody and read out at a scanning intensity of 7 (red; FIGS. 3A-B). A very strong antibody response against the peptide having the sequence as set forth in SEQ ID NO: 97 (which comprises the motif GRSNTL (SEQ ID NO: 4)) was found.

Mouse Antibody Mono 26:

Incubation of mouse antibody Mono 26 at concentrations of 10 μg/ml, 100 μg/ml (scans not shown) and 250 μg/ml was followed by staining with secondary goat anti-mouse IgG (H+L) DyLight680 antibody and read out at a scanning intensity of 7 (red, FIG. 4A). Moderate monoclonal antibody response against clear epitope-like spot patterns formed by adjacent peptides with the consensus motif KENTKND (SEQ ID NO: 98) with all three peptide lengths (FIG. 4 B). The core sequence which showed the strongest binding was NTKN (SEQ ID NO: 74).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An antibody comprising an antigen recognition domain that binds specifically the extracellular domain of TNF-related apoptosis-inducing ligand (TRAIL) between amino acids 95-155 and/or amino acids 190-210, with the proviso that the antibody is not an antibody set forth in Table 2.

2. The antibody of claim 1, wherein said antigen recognition domain binds said TRAIL between amino acids 130-140 and/or 195-205.

3. An antibody comprising an antigen recognition domain that binds specifically to at least one epitope of TNF-related apoptosis-inducing ligand (TRAIL), wherein said at least one epitope is in an amino acid sequence selected from the group consisting of SEQ ID NOs: 1-95, 97, 98 and 101-109, with the proviso that the antibody is not an antibody set forth in Table 2.

4. The antibody of claim 1, wherein said antigen recognition domain binds to at least one epitope which is in an amino acid sequence selected from the group consisting of SEQ ID NO: 1-8, 10-95 and 101-108.

5. The antibody of claim 1, wherein said antigen recognition domain binds to at least four contiguous amino acids of SEQ ID NO: 15 (FRFQEEIKENTKND) or SEQ ID NO: 52 (ITGTRGRSNTLSSPNSK).

6. The antibody of claim 1, being a monoclonal antibody.

7. The antibody of claim 6, wherein said at least one epitope is in the amino acid sequence as set forth in SEQ ID NO: 4 or 74.

8. The antibody of claim 1, capable of binding said TRAIL when presented in a blood sample at a concentration below 70 μg/ml, as determined by an immunoassay.

9. The antibody of claim 6, being of an IgG subclass.

10. The antibody of claim 1, comprising a detectable moiety.

11. An isolated polynucleotide comprising a nucleic acid sequence encoding the antibody of claim 6.

12. A method of generating a TRAIL antibody comprising immunizing a non-human animal with a peptide having an amino acid sequence selected from the group consisting of SEQ ID NO: 1-95, 97, 98 and 101-109, wherein the peptide is no longer than 20 amino acids, thereby generating the TRAIL antibody.

13. The method of claim 12, further comprising isolating said TRAIL antibody.

14. The method of claim 13, further comprising analyzing the affinity of said TRAIL antibody to TRAIL.

15. A method of generating a TRAIL antibody comprising expressing the polynucleotide of claim 11 in a cell, thereby generating the antibody.

16. The method of claim 15, further comprising isolating the antibody.

17. A composition of matter comprising a carrier and a peptide which is no longer than 20 amino acids, wherein said peptide comprises an amino acid sequence selected from the group consisting of SEQ ID NO: 1-95, 97, 98 and 101-109, said carrier being selected from the group consisting of bovine serum albumin (BSA), keyhole limpet hemocyanin (KLH), ovalbumin (OVA) and poly-Lys.

18. A method of detecting TRAIL in a biological sample comprising:
(a) contacting the sample with the antibody of claim 1; and
(b) detecting said antibody.

19. The method of claim 18, wherein the antibody is a monoclonal antibody.

20. The method of claim 19, wherein said detecting is effected using a second antibody.

21. The method of claim 20, wherein said second antibody binds to said TRAIL at a different epitope to the antibody used in step (a).

22. The method of claim 20, wherein said second antibody binds to said TRAIL at an epitope that overlaps the epitope of the antibody used in step (a).

23. The method of claim 19, wherein said monoclonal antibody is attached to a solid support.

24. A composition of matter comprising blood and the antibody of claim 1.
